(19) United States
(12) Patent Application Publication
(10) Pub. No.: US 2005/0096286 A1
(43) Pub. Date: May 5, 2005

(54) Treatment of hypercholesterolemia or diabetes associated angiogenic defects

(75) Inventors: Alexis Caron, La Hay les Roses (FR); Florence Emmanuel, L'lle Saint Denis (FR); Francoise S. Finiels, Chennevieres sur Marne (FR); Sandrine Michelet, Pontault-Combault (FR); Anne Caron, Montrouge (FR); Didier Rouy, Boust (FR); Didier Branellec, Lyon (FR); Bertrand Schwartz, Autouillet (FR)

Correspondence Address: WILEY, REIN & FIELDING, LLP;ATTN: PATENT ADMINISTRATION, 1776 K. STREET N.W., WASHINGTON, DC, 20006, US

(73) Assignee: Centelion SAS, Vitry sur Seine (FR)

(21) Appl. No.: 10/861,906

(22) Filed: Jun. 7, 2004

Related U.S. Application Data
(60) Provisional application No. 60/475,959, filed on Jun. 5, 2003.
(60) Provisional application No. 60/560,915, filed on Apr. 9, 2004.
(60) Provisional application No. 60/566,193, filed on Apr. 28, 2004.

(30) Foreign Application Priority Data
Jun. 4, 2004 (WO) PCT/EP04/06903

(51) Int. Cl. A61K 48/00

(52) U.S. Cl. 514/44

(57) ABSTRACT

The present invention relates to the use of a plasmid encoding a fibroblast growth factor as therapeutic agent for the prevention and treatment of hypercholesterolemia or diabetes associated myocardial or skeletal angiogenic defects. The present invention also relates to a method for enhancing formation of both collateral blood vessels and arterioles in myocardial or skeletal ischemic tissues in a mammalian subject suffering from hypercholesterolemia or diabetes. The present invention further relates to a method of promoting collateral blood vessels in ischemic myocardial or skeletal tissues without inducing VEGF-A factor expression and causing edema in the treated muscles.

### FIELD OF THE INVENTION AND INTRODUCTION

The present invention relates to the use of a plasmid encoding a fibroblast growth factor as a therapeutic agent for the prevention and treatment of hypercholesterolemia or diabetes associated myocardial or skeletal angiogenic defects. The present invention also relates to a method for enhancing formation of both collateral blood vessels and arterioles in myocardial or skeletal ischemic tissues in a mammalian subject suffering from hypercholesterolemia or diabetes. The present invention further relates to a method of promoting collateral blood vessels in ischemic myocardial or skeletal tissues without inducing VEGF-A factor expression and causing edema in the treated muscles.

### BACKGROUND OF THE INVENTION

The blood vessels form a closed blood delivery system that begins and ends at the heart, which comprises three major types of blood vessels, i.e., arteries, capillaries, and veins. As the heart beats, blood is forced into the large arteries from the ventricles. The large arteries branch into medium-sized arteries, which branch into smaller arteries that deliver blood to various parts of the body. The arteries divide again and again until they reach their smallest branches, the arterioles. As arterioles enter tissue, they branch into the microscopic vessels called capillaries, which lie close to tissue cells. The capillaries have very thin walls. Oxygen and nutrients leave the blood in the capillaries and enter the tissue cells, and carbon dioxide and other wastes leave the cells and enter the blood within the capillaries. Before the capillaries leave the tissue, they merge to form small veins called venules. The venules merge to form progressively larger veins that ultimately empty into the great veins that return blood to the heart.

The walls of all blood vessels, except capillaries, are composed of 3 distinct layers surrounding the lumen. The innermost layer that lines the vessel lumen is called the tunica interna, and consists primarily of endothelium cells. The middle layer, the tunica media, consists mostly of circularly arranged smooth muscle cells. The outermost layer of the blood vessel wall, the tunica extema, is composed mostly of elastic fibers and collagen fibers that protect the blood vessel and anchor it to surrounding structures. The tunica extema is infiltrated with nerve fibers and, in the larger arteries and veins, a system of tiny blood vessels.

Arterioles are the smallest arteries and have a lumen diameter smaller than 50 μm. The wall of the arteriole consists of the tunica interna surrounded by scattered smooth muscle fibers in the tunica media. Arterioles regulate blood flow from arteries into capillaries. During vasoconstriction of arteriole walls, blood flow into capillaries is restricted and the tissues served by the arteriole may be momentarily bypassed. During vasodilatation of arteriole walls, blood flow into the capillaries increases significantly.

In contrast, capillaries have extremely thin walls, which only consist of one line of endothelial cells—just the tunica interna. They form extensive networks that permeate nearly all body tissues and almost every cell of the body. The average lumen diameter of a capillary is 0.01 mm (10 μm), just large enough for red blood cells to slip through in single file. The extremely thin walls make the capillaries perfectly suited for their purpose, which is the exchange of nutrients, oxygen and waste products with the cells of the body.

Collateral blood vessels play a significant role in supplying oxygen to an organ, particularly when oxygen delivery is limited by disease in the normal vasculature. Collateral vessels can be pre-existing vessels that normally have little or no blood flow. Acute occlusion of normal vessels (e.g., thrombosis of a large artery) can cause a redistribution of pressures within the vascular bed, thereby causing blood flow to occur in collateral vessels. Collateral blood vessels are particularly important in the coronary and skeletal muscle (e.g., human leg) circulations. In the heart, collateral vessels can help to supply blood flow to ischemic regions due to stenosis or occlusion of epicardial arteries. Collateral blood flow may be an important mechanism in limiting infarct size. Formation of collateral blood vessels is triggered in the therapeutic angiogenesis.

Angiogenesis is a complex process which involves proliferation of endothelial cells, the degradation of the basement membrane, the migration through the surrounding matrix, as well as the alignment and differentiation into tube-like structures to form the walls of blood vessels, thus resulting in a newly formed capillary network.

Arteriogenesis, which refers to the outgrowth of collateral arterioles, is also believed to be the most efficient process for restoration of blood perfusion because of the high capacity of these vessels compared with the capillary network (Carmeliet et al., Nat. Med., 2000; 6:389-395; Van Royen et al., Cardiovasc. Res., 2001;49:543-553). In effect, arterioles are considered as mature, robust and functional vessels due to the presence of both tunica interna and media, i.e., a layer of endothelial cells supported by a layer of smooth muscle cells. Formation of arterioles is a preferred type for long term and effective neovascularization.

Among the pathological conditions associated with a vascular endothelial dysfunction is hypercholesterolemia, a disease characterized by abnormal vessel formation, an impaired regulation of tissue perfusion, abnormal spatial distribution of blood flow, as well as abnormal microvascular function. Also, kinetics of vessel growth as well as the nature of resultant vessels are different from healthy tissues. These changes can be the result of impaired vascular endothelium, which shows a reduced signal transduction, a reduced availability of L arginine, a reduced expression of eNOS, NO (nitrous oxide) inactivation increased by superoxyde anion derived from macrophages or other inflammatory cells, release of several vasoconstricting factors, such as endothelin, and smooth muscle vascular response. In subjects with hypercholesterolemia, capillary density and distribution in the arterial wall change dramatically. Ultimately, the changes show dense plexi of adventitial microvessels with marked disorientation. It is believed that this may result from different stimuli that may cause stronger or weaker angiogenic response. Hypercholesterolemia in humans causes a vascular endothelial dysfunction and ultimately a progressive narrowing of the main arteries. The unbalance in the coronary blood flow at rest and during stress creates a furtive malfunction of the muscle that leads to pain and hypocontractility. Usually the supplies are appropriate at rest, but when stress occurs, the needs increase while the supplies may be further reduced due to the arterial obstructive lesions. Mimicking this human pathology leads to both an assay related to the setup of a stenosis, for example, on a major coronary artery, and the use of a stress test to reveal the unbalance created at stress by this stenosis.

It is also known that vascular function in patients with types I and II diabetes mellitus is characterized by impaired endothelium-dependent relaxation. Diabetes mellitus is characterized by premature development of microvascular and macrovascular disease (Kannel et al., Diabetes Care, 1979, 241:2035-2038). It is not known whether conditions of severe endothelium impairment and abnormalities due to hypercholesterolemia, diabetes, hypertension and hyperlipidemia in patients suffering from peripheral arterial disease (PAD), peripheral arterial occlusive disease (PAOD), or cardiac artery disease (CAD) might be improved or rescued by using therapeutic angiogenesis.

Administration of several angiogenic factors such as, for example, VEGF, aFGF and bFGF, HIF-1αVP16 and TGF-β to promote collateral blood vessels, also known as therapeutic angiogenesis, has been proposed for the treatment of ischemic cardiovascular and peripheral ischemia. However, the potent pleiotropic effects on various cell types may limit the therapeutic applicability of some of these compounds. For example, VEGF-A was one of the most potent candidate angiogenic factors. However, VEGF-A was shown to generate edema, as well as disorganized, tortuous and leaky vessels, resembling those found in tumors (Lee et al., Circulation 2000, 102:898-901; Springer et al., Mol Cell, 1998,2:549-559).

Two distinct strategies for therapeutic angiogenesis exist. Protein therapy, which involves delivery of the growth factor directly into the ischemic tissue, is a possible option. Angiogenic gene therapy is an alternative option aimed at improving collateral development and overcoming perfusion defects and related ischemia through the transfer of nucleic acids to somatic cells (8-10).

Animal studies have demonstrated the local angiogenic potential of many growth factors including, for example, recombinant human VEGF, recombinant PDGF, or recombinant bFGF. However, delivery of recombinant proteins, and the systemic administration of high doses of recombinant proteins was shown to lead to a multitude of other negative side-effects. Furthermore, the quantity of the recombinant protein required is important. If too little protein is delivered, angiogenesis will not be achieved. If too much protein is delivered, the formation of disorganized vasculature beds and promiscuous angiogenesis can result.

Therapeutic angiogenesis via the administration of nucleic acids (either in a naked form or via liposomes or viral vectors) capable of expressing an angiogenic protein has been investigated. Viral vector delivery of an angiogenic coding sequence allows for high efficiency of delivery, but suffers from numerous disadvantages related to viral vectors, such as the occurrence of an immune reaction as well as the possibility of integration and dissemination. For example, adenovirus gene therapy methods have been questioned following the death of Jesse Gelsinger in September 1999 at the University of Pennsylvania after receiving, through intrahepatic artery infusion, E1- and E4-deleted recombinant adenovirus, which expressed a correct form of the human omithine transcarbamylase. Pathological analyses have indicated that the official cause of death was a multi-organ failure secondary to adult respiratory distress syndrome induced by a systemic inflammatory response to recombinant adenovirus administered systematically. More recently, two cases of leukemia have been reported in a trial aimed at treating children with severe combined immunodeficiency disease (SCID), which were attributed to the use of retrovirus as a vector.

Both liposomes and naked DNA comprising a DNA encoding an angiogenic peptide also suffer from a major disadvantage, which is a lesser efficiency of delivery when compared to virus; thus the level of the protein needed to achieve a therapeutic effect may be difficult to reach.

Relating to naked DNA strategy, it was surprisingly demonstrated that intramuscular injection of NV1FGF, a plasmid encoding an acidic Fibroblast Growth Factor or Fibroblast Growth Factor type 1 (FGF-1), for patients with end-stage peripheral arterial occlusive disease (PAOD) or with peripheral arterial disease (PAD), meets safety requirements. Camerota et al. (J Vasc. Surg., 2002, 35, 5:930-936) describes that 51 patients with unreconstructible end-stage PAD, with pain at rest or tissue necrosis, have been intramuscularly injected with increasing single or repeated doses of NV1FGF into ischemic thigh and calf. Various parameters such as transcutaneous oxygen pressure, ankle and toe brachial indexes, pain assessment, and ulcer healing have been subsequently assessed. A significant increase of brachial indexes, reduction of pain, resolution of ulcer size, and an improved perfusion after NV1FGF administration were observed.

Induction of angiogenesis by VEGF gene transfer in patients with hindlimb ischemia has also been demonstrated. Naked plasmid DNA encoding VEGF-165 isoform was administered in ischemic muscles of patients with non-healing ischemic ulcers and/or pain at rest due to peripheral arterial disease. Newly developed collateral blood vessels and improved perfusion could be seen angiographically at 8 weeks post-treatment as well as capillaries. However, a significant elevation of the serum levels of VEGF at 5 to 6 weeks after treatment was also observed (Isner et al., Lancet, 1996; 348:370-4). Such elevation of the serum VEGF level may cause promiscuous unwanted angiogenesis, and serious negative side effects such as edema (26).

Vincent et al., (Circulation, 2000; 102(18):2225-61) report that administration of naked DNA plasmid encoding for the Hypoxia Induced Factor 1α (HIF-1α) transcription factor was associated with significant improvements in calf blood pressure ratio, angiogenic score, regional blood flow and capillary density. However, it is also reported that HIF-1α activates expression of endogenous VEFG gene suggesting the enhancement of VEGF-pathway dependant angiogenesis as well as several targets in vivo.

Taniyama et al., (Gene Therapy, 2001, 8: 181-189) have further reported therapeutic angiogenesis using intramuscular injection of naked DNA plasmid coding for a human Hepatocyte Growth Factor (HGF) in rat and rabbit ischemic hindlimb models. An increase of the collateral blood vessels was identified by angiography and capillary density as demonstrated by alkaline phosphatase as a marker of endothelial cells. HGF which was first identified as a mitogen for hepatocytes, has also been shown to be a mitogen for certain cell types including melanocytes, renal tubular cells, keratinocytes, and certain endothelial cells and cells of epithelial origin (Matsumoto et al., BBRC, 1991, 176:45-51). HGF was also shown to stimulate growth of endothelial cells without replication of vascular smooth muscle cells (Nakamura et al., Hypertension, 1996; 28:409-413; Hayashi et al., BBRC, 1996; 220:539-545). Finally, it was shown that HGF can also act as a “scatter factor,” an activity that promotes the dissociation of epithelial and vascular endothelial cells (Giordano et al., PNAS, 1993, 90:649-653). Therefore, HGF has been postulated to be involved in tumor formation.

In contrast, administration of a plasmid encoding acidic fibroblast growth factor (aFGF or FGF-1) was proved not to increase the FGF-1 serum level, thereby showing that the use of such human FGF-1 expression plasmid is particularly advantageous in terms of safety as promiscuous angiogenesis or negative side effects are absent. The absence of circulating FGF-1 provides a significant safety advantage over other angiogenic factors such as, for example, VEGF or FGF-2, which have been described to leak into the circulation and lead to distant edema (Baumgartner et al., Circulation, 1998, 97:1114-23).

The fibroblast growth factor (FGF) family is comprised of at least 23 structurally related proteins (FGF 1-23) whose best known members are FGF-1, FGF-2, FGF-4, FGF-7 and FGF-9. Members of this family stimulates late mitogenesis in most cells derived from mesoderm and neuroectoderm and influence other biological processes, including angiogenesis, neurite extension, osteoblast growth, neuronal survival, and myoblast differentiation. In general, FGFs have a high affinity for heparin. Prior to resolution of their nomenclature, some FGFs were referred to as heparin-binding growth factors-1, -2, etc., and many, but not all, are mitogens for fibroblasts. The members of the FGF family possess roughly 25-55% amino acid sequence identity within a core sequence and some FGFs possess significant extensions, either C-terminal, N-terminal, or both, outside of this core sequence. This structural homology suggests that the different genes encoding known FGFs may be derived from a common, ancestral gene.

In addition to the 23 known members of the FGF family, additional complexity results from the generation of several molecular forms of FGF from a single gene. For example, the primary translation product of aFGF (FGF-1) consists of 155 residues. However, the longest form of FGF-1 found in a natural source (e.g., bovine brain) consists of 154 residues. This 154 residue form of FGF-1 lacks the NH2-terminal methionine of the 155 residue form and has an acetylated amino terminus. Proteolytic processing in vivo or during purification generates smaller active forms of FGF-1 in which either the amino-terminal 15 (des 1-15) or 21 (des 1-21) amino acids are deleted. As defined herein, FGF-1 refers to the 154 residue form of FGF-1 and shorter, biologically active forms thereof, such as the above described forms deleted of the amino-terminal 15 (des 1-15) or 21 (des 1-21) amino acids. Historically, the 154 residue form of FGF-1 was termed 13-endothelial cell growth factor (β-ECGF), the des 1-15 form was termed aFGF or FGF-1, and the des 1-21 form was termed .alpha.-ECGF. Prior to standardization of the terminology for this group of growth factors, several additional terms were also applied to the same protein, including eye derived growth factor and heparin binding growth factor 1. Similar forms of bFGF (FGF-2) have also been described. In addition to cleaved forms, extended forms of bFGF have also been described, resulting from initiation of translation at several different GTG codons located upstream of the ATG translation initiation codon which generates the 155 residue form of bFGF. All of these alternative forms of the FGFs contain the core region of structural homology which defines the FGF family. Many of the various FGF molecules have been isolated and administered to various animal models of myocardial ischemia with varying and often times opposite results.

An angiogenic role for FGF-1 was suggested based on in vivo studies (Comerota et al., J. Vasc. Surg., 2002, 35, 5:930-936). Intramuscular injections of FGF-1 expression plasmid demonstrated an improved perfusion based on an increased in ankle brachial index, reduction in pain, and an increased transcutaneous oxygen.

The Applicant has now surprisingly discovered that intramuscular injection of an FGF-1 expression plasmid does not cause induction of VEGF expression in vascular endothelial cells, and thus constitutes a very safe angiogenesis therapy in contrast with other angiogenic factors, including other FGF factors, VEGF, HIF-1α/VP16 and HGF.

Most therapeutic angiogenesis studies have been validated in animal models of limb ischemia and performed in normal healthy animals, but few have been tested for their capacity to reverse angiogenesis defects in a hypercholesterolemia or diabetes setting, wherein endothelium function is greatly impaired.

In this regard, known therapeutic angiogenesis treatments have not proven to be convincing when tested in the hypercholesterolemic rabbit model subjected to femoral artery excision, as an impaired collateral vessel formation and capillary density that could be only partially reversed by administration of VEGF was observed (26). In addition, therapeutic angiogenesis were not proved to be convincing when tested in diabetes models, as Roguin A. et al. (Cardiovascular Diabetology 2003, 2:18) showed that use of VEGF expression plasmid failed to improve blood flow and to promote collateralization in a diabetic ischemic mouse.

In contrast, the Applicant has surprisingly found that a plasmid expressing the human FGF-1 when administered intramuscularly in ischemic myocardial or skeletal muscles was capable of efficiently reversing the hypercholesterolemia or diabetes associated defects in collateral vessels and promoting the formation of mature vessels such as arterioles in a mammalian subject suffering from hypercholesterolemia or diabetes.

In addition, the applicant has discovered that in contrast with similar angiogenic factors, the FGF-1 expression plasmid intramuscular injection did not cause edema in the treated skeletal or cardiac muscle and thus could be used in an amount sufficient to rescue angiogenesis defects of ischemic muscles in aggravated conditions such as hypercholesterolemia or diabetes setting.

### SUMMARY OF THE INVENTION

The present invention concerns a method for treating myocardial or skeletal angiogenic defects associated with hypercholesterolemia or diabetes comprising the administration to the subject of pharmaceutical compositions comprising a plasmid carrying a gene encoding a fibroblast growth factor in an amount which promotes reversal of endothelium dysfunction and angiogenic defects.

The present invention also relates to a method of treating myocardial or skeletal angiogenic disorders or defects associated with hypercholesterolemia or diabetes comprising administering an effective amount of a plasmid encoding a fibroblast growth factor, wherein VEGF-A factor expression is not induced in the myocardial or skeletal muscle.

The present invention further concerns a method of treating vascular endothelium dysfunction associated with hypercholesterolemia or diabetes in a patient via the administration in skeletal or myocardial muscles of an amount of a plasmid encoding a fibroblast growth factor sufficient to reverse myocardial or skeletal angiogenic defects, wherein VEGF-A factor expression is not induced and an edema is not generated.

It is also an object of the present invention to provide a method of stimulating and/or promoting the formation of mature collateral vessels in ischemic cardiac or skeletal muscle tissues in ischemic muscles in a hypercholesterolemia or diabetes setting, comprising injecting said tissues of said subject with an effective amount of a plasmid encoding a fibroblast growth factor, wherein VEGF-A factor expression is not induced and/or an edema is not generated.

Another object of the present invention is to provide a method for treating ischemic conditions such as PAD, PAOD or CAD in a mammalian subject suffering from hypercholesterolemia or diabetes, without inducing expression of the VEGF factor, and without causing formation of edema.

Still another object of the present invention is to provide a method for promoting both collateral blood vessels and arterioles in ischemic tissues, wherein endothelial function is impaired.

A further object is a method for reversing angiogenesis defects elicited by hypercholesterolemia or diabetes, without inducing expression of the VEGF factor in a mammalian subject in need for such treatment suffering from hypercholesterolemia or diabetes.

Still a further object of the present invention is to provide a method of promoting angiogenesis VEGF-independent pathway.

A further object of the present invention is to provide a method of promoting angiogenesis with the provisio that VEGF is not upregulated in the treated cells.

Intramyocardial or intramuscular injection of the FGF expression plasmid is preferably for the reversal of myocardial or skeletal angiogenic defects associated with hypercholesterolemia or diabetes. The fibroblast growth factors preferred in the practice of the present invention is FGF-1, and most preferably the human full-length FGF-1.

Other and further objects, features and advantages will be apparent from the following description of the preferred embodiments of the invention given for the purpose of the disclosure when taken in conjunction with the following drawings.

### BRIEF DESCRIPTION OF THE FIGURES

FIG. 1: is a schematic of the design of the experiments.

FIGS. 2(A)-(C): represent cross-sections (magnification ×100) of hamster muscles (Gracillis and Adductores) after HES staining; FIG. 2(A) is a cross-section of non- ischemic (contralateral) muscles; FIG. 2(B) and (C) are cross-sections of ischemic muscles; dashed lines in FIG. 2(B) illustrates the presence of mild necrosis; the arrow in FIG. 2(C) points to centronucleation.

FIGS. 3(A)-(C): set forth representative angiograms recorded in both non-ischemic (left) and ischemic (right) hindlimbs from hamsters of LC (A); HC/21 (B); and HC/28(C).

FIG. 3(D): illustrates the corresponding angiographic score obtained by quantification of collateral formation after hindlimb ischemia.

FIGS. 4(A)-(D): show representative cross-sections (magnification ×100) of mature vessels labeled by smooth muscle α-actin (SMA) immunohistochemistry from non- ischemic (A and C) and ischemic (B and D) muscles (Adductores and Gracilis) harvested at day 21 (A and B) or at day 28 (C and D) after induction of ischemia.

FIGS. 4(E) and (F): represent a graph illustrating quantification of muscle area and <50 μm SMA-positive arterioles. Empty bars: non-ischemic hindlimb; Hatched bars: ischemic hindlimb. NS: not significant; **: p<0.01 vs. any group.

FIGS. 5(A) and (B): set forth representative angiograms of both non-ischemic and ischemic hindlimbs from hamsters treated with saline (A) or NV1FGF (B).

FIG. 5(C): illustrates quantification of collateral formation through angiographic score seen after hindlimb ischemia.

FIGS. 6(A) and (B): display representative cross-sections (magnification ×100) depicting mature vessels labeled by smooth muscle α-actin (SMA) immunochemistry from ischemic muscles (Adductores and Gracilis) of hamsters treated with saline (A) or NV1FGF (B).

FIGS. 6(C) and (D): represent graphs showing quantification of muscle area and <50 μm SMA-positive arterioles. Empty bars: non-ischemic hindlimb; Hatched bars: ischemic hindlimb. NS: Not significant; *: p<0.01; ** p<0.01 saline vs. NV1FGF.

FIG. 7: are representative pictures (magnification ×100) of immunochemical staining with an anti-FGF-1 polyclonal antibody in muscles from the back part of the thigh (Gracilis and Adductores) from non-ischemic (controlateral) on injected limbs (A) and ischemic limbs injected with saline (B) or NV1FGF (C). Arrows show immunoreactive fibers identified by the brown staining of the immune complexes.

FIGS. 8(A)-(C): are histological sections from Tibialis Cranialis muscles stained by antibody to murine VEGF. (A): NaCl injected muscle section with a mosaic aspect of myofiber positivity. (B): pCOR-CMV-empty injected muscle section with a similar aspect. (C) NaCl injected muscle section after adsorption of antibody to mVEGF-A with mVEGF-A peptide.

FIGS. 8(D) and (E): are histological serial sections of pCOR-CMV.rat-spFGF-1 injected Tibialis Cranialis muscle stained by antibody to mVEGF-A (D) or FGF-1 (E).

FIG. 9A: displays a template showing the location of the 13 injections in the heart of the rabbit with hypercholesterolemia, where X=injection site.

FIG. 9B: is a microscopic photo of HES stained sections of the left circumflex coronary artery from hypercholesteromic Watanabe rabbit at a magnification of ×100. A corresponds to Adventicia; M corresponds to Media; Arrow heads correspond to Intima; Arrow corresponds to atherosclerotic plaque;

FIG. 10: displays an ECG at rest in humans according to the nomenclature of the ST segment modifications during a stress test in humans (from Braunwald et al. Heart Disease, 5^{th} ed. p159). On the left of FIG. 10 is shown the ECG at rest in humans, and on the right, from top to bottom, a progressively more serious modification of the ST segment, depending on the slope of this segment: upsloping, horizontal, and downsloping. The elevation shown at the bottom is the most serious.

FIG. 11A: displays an ECG in rabbits during a dobutamin stress test.

FIG. 11B: displays an enlargement of the lead I. The ST depression, horizontal is clearly seen just after the QRS complex by a large vertical peak.

FIG. 12: displays an ECG scoring at the highest dobutamin dose

FIG. 13A: displays a typical 12 lead ECG in a rabbit at rest.

FIG. 13B: displays a strong ischemia at maximum stress in the same rabbit.

FIG. 14: is a schematic of the nomenclature of the 2D echocardiography.

FIGS. 15: show myocardial microscopic lesions and associated FGF-1 expression in healthy rabbit heart 3 days after the injection of NV1FGF. HES staining demonstrating myocardial degeneration and necrosis with active chronic inflammatory response (A) and associated FGF-1 expression (arrow heads, B). Background (*) is relative to secondary antibody conjugation (anti-rabbit) with endogenous IgG. Magnification: ×100.

FIG. 16: displays the evolution of the maximum ECG score during the stress test on rabbits treated with empty plasmid (grey column) or NV1-FGF plasmids (hatched column).

FIG. 17: displays the quantification of 16 normal segments (grey) and 14 abnormal segments (black). The qualification normal/abnormal was a visual evaluation.

FIG. 18: displays a plot of the ECG maximum score versus the Echo maximum score. The regression curve is shown in black. The two main zones (abnormal ECG and abnormal echo, normal ECG and normal echo) are shaded in grey.

FIG. 19: displays the evolution of the echocardiographic score with the time after treatment. The NV1-FGF treated animals are shown in hatched, the empty plasmid-treated animals are shown in grey. A * indicates a significant difference (p<0.05) between groups.

FIG. 20: presents a standardized procedure used for the preparation of heart sections samples for histologic analysis with various sectors of the heart according to the 3 short axis slices, e.g., apical, mid and basal segments.

FIG. 21: displays a quantitative analysis of vascular density in the scar in viable myocardium distant from the scar.

### DETAILED DESCRIPTION OF THE INVENTION

The present invention provides, inter alia, a method for treating or repairing myocardial or skeletal angiogenic defects associated with hypercholesterolemia or diabetes in which endothelial functions are impaired or inadequate.

The present method and composition are particularly useful in reversing endothelium dysfunction associated with hypercholesterolemia or diabetes, following direct intramuscular administration to promote a net increase of blood vessel formation in the myocardial or skeletal muscle. The invention encompasses the use of a plasmid encoding a biologically active fibroblast growth factor and pharmaceutically acceptable salts and derivatives thereof.

The present invention also provides a method of promoting the formation of mature collateral vessels in ischemic cardiac or skeletal muscle tissues in a mammalian subject in need of such treatment comprising injecting said tissues of said subject with an effective amount of a plasmid encoding a fibroblast growth factor, wherein VEGF-A factor expression is not induced in said subject. Particularly, administration of FGF expressing plasmid induces the formation of both collateral blood vessels and arterioles in ischemic myocardial or skeletal muscle tissues, without inducing expression of the VEGF-A factor. A particular advantage of the inventive methods using the FGF expression plasmid according to the present invention is that they do not cause side effects such as edema.

The present invention further provides a method of reversing defects in angiogenesis elicited by hypercholesterolemia or diabetes, without inducing VEGF-A factor expression, and/or causing the formation of edema, comprising injecting myocardial or skeletal tissues of said patient with an effective amount of a plasmid expressing a fibroblast growth factor to promote the formation of both collateral blood vessels and arterioles.

The present invention further provides a method for enhancing revascularization by promoting both collateral blood vessels and arterioles in ischemic tissues of a mammalian subject in a hypercholesterolemic or diabetes setting, which comprises injecting said tissues of said subject with an effective amount of a FGF expression plasmid to reverse angiogenesis defects. The delivery and expression of said plasmid unexpectedly results in a significant improvement of the blood perfusion throughout the ischemic muscles.

The term “subject” includes, but is not limited to, mammals, such as dogs, cats, horses, cows, pigs, rats, mice, simians, and humans.

The term biologically active sequence means a nucleotide sequence encoding a naturally occurring peptide or any biologically active analogues or fragments thereof. Different forms exist in nature with variations in the sequence of the structural gene coding for peptides of identical biological function. These biologically active sequence analogues include naturally and non-naturally occurring analogues having single or multiple amino acid substitutions, deletions, additions, or replacements. All such allelic variations modifications and analogues resulting in derivatives which retain one or more of the native biologically active properties are included in the scope of this invention.

The FGF encoding plasmid thus comprises a nucleotide sequence that encodes the desired FGF protein. These molecules may be cDNA, genomic DNA, synthesized DNA or a hybrid thereof or an RNA molecule such as mRNA. Preferably, the plasmid comprises a nucleotide sequence encoding the FGF-1 and thus encompasses a nucleotide sequence encoding the 154 residue form of FGF-1 acidic growth factor as described in U.S. Pat. No. 4,686,113.

The regulatory elements necessary for gene expression of a DNA molecule may comprise a promoter, an initiation codon, a stop codon, and a polyadenylation signal. In addition, enhancers are often desired for gene expression. It is necessary that these elements be operably linked to the sequence that encodes the desired proteins and necessary or preferred that the regulatory elements are operable in the myocardium of the subject to whom they are administered.

Initiation and stop codons are generally considered to be part of a nucleotide sequence that encodes the desired protein. However, it is necessary that these elements are functional in the subject to whom the gene construct is administered. The initiation and termination codons must be in frame with the coding sequence.

Promoters and polyadenylation signals used must be functional within the myocardial cells of the subject.

Examples of promoters useful to practice the present invention include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human alpha actin, human Myosin, human Hemoglobin, human muscle creatine and human metalothionein. One skilled in the art is familiar with many promoter and regulatory elements that can similarly be used with the invention.

In another preferred embodiment, the expression of the FGF genes is driven by a muscle specific promoter, such as the murine or human upstream sequence of the CARP gene, which is described in the US publication 2003/0039984, or the cardiac alpha actin promoter sequence as described in the international publication WO01/11064.

Examples of polyadenylation signals useful to practice the present invention, especially in the production of a genetic vaccine for humans, include but are not limited to SV40 polyadenylation signals, bovine or human Growth hormone polyadenylation signals, and LTR polyadenylation signals. In particular, the SV40 polyadenylation signal which is in pCEP4 plasmid (Invitrogen, San Diego Calif.), referred to as the SV40 polyadenylation signal is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. Plasmids pCEP4 and pREP4 from Invitrogen (San Diego, Calif.) contain the Epstein Barr virus origin of replication and nuclear antigen EBNA-1 coding region which produces high copy episomal replication without integration. Other suitable plasmids are well known to those skilled in the art, for example, plasmid pBR322, with replicator pMB1, or plasmid pMK16, with replicator ColE1 (Ausubel, Current Protocols in Molecular Biology, John Wiley and Sons, New York (1988) §II:1.5.2.

In a preferred embodiment, the FGF encoding plasmid is present in a conditional origin of replication pCOR plasmid as described in the International application WO 97/10343, WO 03/03373, and Soubrier et al. (Gene Ther. 1999;6:1482-1488). The pCOR plasmid harbors an optimized expression cassette encoding a secreted form of human FGF-1 (sphFGF-1) inserted into an original backbone. The resulting plasmid is advantageously of small size of 2.4 kb. The sequence encoding sphFGF-1 is a fusion between the sequences encoding the secretion signal peptide (sp) from human fibroblast interferon and the naturally occurring truncated form of human FGF-1 from amino acids 21 to 154 (U.S. Pat. No. 4,686,113; U.S. Pat. No. 5,849,538). Expression of sphFGF-1 is driven by the human cytomegalovirus (CMV) immediate early enhancer/promoter (from nucleotide −522 to +72). The late polyadenylation signal from simian virus 40 (nucleotides 2538 to 2759 from SV40 genome, GenBank locus SV4CG; U.S. Pat. No. 5,168,062) is inserted downstream of the sphFGF-1 fusion to ensure proper and efficient transcription termination and subsequent polyadenylation of the sphFGF-1 transcript. This preferred plasmid is designated NV1FGF and is devoid of any antibiotic resistance gene.

Plasmid selection relies on a suppressor transfer RNA gene in the autotrophic recipient strain. Maintenance of high copy number and strictly limited host range of the plasmid were obtained with the R6K γ origin of replication. The sequence coding for this protein is not usually found in bacteria but is artificially inserted into the genome of the selected host strain. Thus, plasmid dissemination is greatly limited.

Plasmids according to the present invention can be administered to a vertebrate by any method that delivers injectable materials to cells of the myocardium. Preferably, the plasmids are administered as naked DNA plasmid in the sense that they are free from any delivery vehicle that can act to facilitate entry into the cell, for example, the polynucleotide sequences are free of viral sequences, particularly any viral particles which may carry genetic information. They are similarly free from, or naked with respect to, any material which promotes transfection, such as liposomal formulations, charged lipids such as Lipofectin™, or precipitating agents such as CaPO₄. Plasmid may otherwise be delivered to the animal with a pharmaceutically acceptable liquid carrier, as known in the art. In preferred applications, the liquid carrier is aqueous or partly aqueous, comprising sterile, pyrogen-free water. The pH of the preparation is suitably adjusted and buffered. Alternatively, the plasmid may be injected with the use of liposomes, such as cationic or positively charged liposomes.

The following Examples clearly demonstrate that a plasmid, here NV1FGF, allows a slow release of the encoded FGF-1 protein at a concentration sufficient to promote a sustained angiogenic response via the formation of capillary vessels as well as mature vessels such as arterioles. Of course, other plasmids can be used for expression of a fibroblast growth factor or FGF-1. In addition NV1FGF was shown to be particularly potent, as it was demonstrated to efficiently promote angiogenesis at a non-detectable concentration in treated muscles. NV1FGF may thus be used at concentrations which are within a therapeutic window, thereby avoiding negative side effects due to dissemination to surrounding tissues or organs or promiscuous angiogenesis.

In addition, it was demonstrated that due to such superior characteristics in terms of safety and potency, NV1FGF was particularly useful for therapeutic angiogenesis in aggravated conditions caused by hypercholesterolemia or diabetes.

The reversal or removal of angiogenesis defects caused by attenuated blood supply regardless of its origin, which is aggravated in conditions such as hypercholesterolemia or diabetes, is thus contemplated by the present invention.

Within the context of the present invention, the target tissue thus comprises muscle tissues suffering from or being at risk of suffering from ischemic damage which results when the tissue is deprived of an adequate supply of oxygenated blood, further aggravated in a hypercholesterolemia or diabetes setting. As demonstrated in the Examples, the intramuscular injection of a plasmid, such as NV1FGF, may be efficiently used in a therapeutic window which is compatible with required standard of safety in gene therapy and is capable of inducing angiogenesis in an ischemic tissue further presenting an impaired endothelial function.

According to one embodiment of the present invention, the NV1FGF plasmid is administered in a localized manner to the target muscle tissue. While, any suitable means of administering the NV1FGF plasmid to the target tissue can be used within the context of the present invention, preferably, such a localized injection to the target muscle tissue is accomplished by directly injecting the NV1FGF to the muscle using a needle.

By the term “injecting” it is meant that the plasmid, such as NV1FGF, is forcefully or intentionally introduced into the target tissue. Any suitable injection device can be used according to the present invention.

While administration of a dose of the NV1FGF plasmid can be accomplished through a single injection to the target tissue, preferably administration of the dose is via multiple injection of NV1FGF. The multiple injections can be 2, 3, 4, 5, or more repeated injections, and preferably 5 or more injections into the ischemic muscle of a mammalian subject suffering from hypercholesterolemia or diabetes. Multiple injections present an advantage over single injections in that they can be manipulated by such parameters as a specific geometry defined by the location on the target tissue where each injection is made. The injection of a single dose of the NV1FGF via multiple injections can be better controlled, and the effectiveness with which any given dose is administered may be maximized.

The specific geometry of the multiple injections may be defined either in two- dimensional space, where the each application of the NV1FGF is administered. The multiple injections may be performed in or around the ischemic tissue, preferably are spaced such that the points of injection are separated by 2 or 3 cm.

According to another embodiment of the present invention, each of the multiple injections is performed within about 5 to 10 minutes of each other.

When administering the NV1FGF to the target tissue which is affected by angiogenesis defects and wherein the endothelium function is severely impaired, it is desirable that the administration is such that the NV1FGF is able to contact a region reasonably adjacent to the source and the terminus for the collateral blood vessel formation, as well as the area therebetween.

Administration of the composition according to the present invention to effect the therapeutic objectives may be by local, intramuscular, parenteral, intravenous, intramyocardial, pericardial, epicardial or via intracoronary administration to the target cardiac muscle tissue. Preferably, intramyocardial, epicardial, pericardial or intracoronary administration is conducted using a needle or a catheter.

Preferably, intramuscular injection of NV1FGF may be performed into the distal thigh and distal leg muscles, and in the region close and surrounding the ischemic site.

Also, when administration is performed by direct intramyocardial injection, those may be performed during an open chest surgery or with the help of a catheter. Catheters for heart delivery are well known in the art and include for example needle catheter as described in U.S. Pat. No. 5,045,565 or 4,661,133, with position sensor system as described in U.S. Pat. Nos. 6,254,573 and 6,309,370. Alternative catheters having a helix needle are described in U.S. Pat. Nos. 6,346,099 and 6,358,247.

In one advantageous aspect of the present invention, a therapeutically effective dose of NV1FGF is administered to reverse the defects in angiogenesis in a hypercholesterolemic or diabetes setting. While the effective dose will vary depending on the weight and condition of a given subject suffering from angiogenesis defects in addition to hypercholesterolemia or diabetic subject, it is considered within the skill in the art to determine the appropriate dosage for a given subject and conditions.

According to a preferred embodiment of this aspect, treatment is performed with dose of about 8000 μg to about 16000 μg of plasmid that is administered by multiple injections of preferably 2 to 4 repeated intramuscular injections of NV1FGF with an interval of time of around 1 to 2 weeks or more, in severe conditions of angiogenesis defects, in order to promote a sustained formation of both collateral vessels and arterioles, thereby allowing to reverse angiogenesis defects due to ischemia in a mammalian subject suffering from hypercholesterolemia or diabetes.

The NV1FGF desirably is administered to the target ischemic muscle in a pharmaceutical composition, which comprises a pharmaceutically acceptable carrier and the NV1FGF plasmid.

Any suitable pharmaceutically acceptable carrier can be used within the context of the present invention, and such carriers are well known in the art. The choice of carrier will be determined, in part, by the particular site to which the composition is to be administered and the particular method used to administer the composition. Formulations suitable for injection include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials, and can be stored in a freeze-dried (lyophilized) condition requiring only the addition of the sterile liquid carrier, for example, water, immediately prior to use. Extemporaneous injection solutions and suspensions can be prepared from sterile powders, granules, and tablets of the kind previously described. Preferably, the pharmaceutically acceptable carrier is a buffered saline solution. Most preferably, the pharmaceutical composition comprises a solution of sodium chloride (0.9%).

In a preferred embodiment the composition of the present invention is administered in association with a low molecular weight heparin (LMWH). LMWH molecules and method of preparation are well-known in the art and are described inter alia in U.S. Pat. No. 5,389,618; U.S. Pat. No. 4,692,435, and U.S. Pat. No. 4,303,651, European patent EP 0 040 144, and by Nenci GG (Vasc. Med, 2000; 5:251-258), which are herein incorporated by reference.

In a second embodiment, the FGF expression plasmid is injected in the skeletal muscle of a hypercholesterolemic or diabetic patient prior or after the administration of an electrical stimulation to the treated skeletal muscle. The electrical stimulation used according to this embodiment is as described in U.S.2002/0031827, and is applied at a voltage and a frequency that do not cause contraction of the skeletal muscle as well as no pain to the patient, as it is below the threshold for muscle contraction. For example, the frequency applied is around 50 Hz, and the voltage is around 0.1 Volt. When used in combination with the FGF expression plasmid, the electrical stimulation results in a synergistic superior effect in terms of increase of blood flow.

In a third embodiment, the FGF-1 expression plasmid is delivered in combination with one or more angiogenesis-promoting factors. Without any limitation, angiogenic factor may include PDGF-AA, PDGF-BB, M-CSF, GMCSF, VEGF-A, VEGF-B, VEGF-C. VEGF-D, VEGF-E, neuropilin, FGF-2(bFGF), FGF-3, FGF-4, FGF-5, FGF-6, Angiopoietin 1, Angiopoietin 2, 1 5 erythropoietin, BMP-2, BMP-4, BMP-7, TGF-beta, IGF-I, Osteopontin, Pleiotropin, Activin, Endothelin-1 and combinations thereof.

Preferably, the NV1FGF is injected in skeletal or cardiac muscle with a PDGFBB expression plasmid and results in a superior formation of collateral blood vessels and arterioles in hypercholesterolemia or diabetes setting. This embodiment thus relates to a method of promoting collateral blood vessels and arterioles comprising delivering NV1FGF and a plasmid expressing PDGF-BB to a localized area of tissue in an amount effective to induce angiogenesis within the area of tissue. The angiogenesis-promoting factor(s) is delivered by expression from isolated DNA encoding the factor following delivery of the DNA to the localized area of tissue.

The present invention also relates to a method of treating PAD and PAOD, CAD or CHF pathologies in patients further suffering from hypercholesterolemia and diabetes.

Impaired perfusion in the hindlimb due to single or multiple large vessel occlusions is the cause of PAD. At an early stage this results in discomfort in the muscles of the leg with ambulation, leading at later stages to ulceration and gangrene (1). Chronic cardiovascular disorders are aggravating factors in patients who are already suffering of ischemic conditions, such as PAD, through mechanisms involving endothelium dysfunction (2, 3). Pathologies such as hypercholesterolemia, hypertension and diabetes have been investigated as possible targets for developing experimental models of PAD (4-7). Nevertheless, in such models of hindlimb ischemia, a critical point is to negate a spontaneous angiogenic response to allow efficacy of any revascularization treatment in an attempt to mimic the clinical situation of hindlimb ischemia.

Genetic models of hypercholesterolemia have also been used to assess angiogenic properties in hypercholesterolemia or diabetes conditions, however in such genetic models a single alteration of a single receptor (deficit in the LDL receptor in Watanabe heritable hyperlipidemic rabbits) or protein (deficit in the glycoprotein ApoE resulting in inceased levels of VLDLs and IDLs in ApoE^{-/-} mice) is generated and do not actually reproduce the conditions of the pathology.

In contrast, NV1FGF plasmid was demonstrated to be particularly potent in reversing hypercholesterolemia-elicited defect in animal models which are very comparable to the pathological conditions found in patients. Indeed, the pathology results from a global lipid overload due to cholesterol-rich diet mimicking the situation encountered in PAD patients suffering from hypercholesterolemia.

According to the present invention, the NV1FGF has been demonstrated to be particularly potent for rescuing cholesterol-induced impairment of angiogenesis in patients suffering from PAD, by promoting the growth of both collateral vessels and arterioles.

Still another object of the present invention is to provide a method for promoting both collateral blood vessels and arterioles in ischemic tissues, wherein endothelial function is impaired.

As shown in the following examples, the NV1FGF is capable to effectively induce the formation of mature large conductance vessels (>150 μm collateral vessels) and small resistance arteries (<50 μm arterioles) in ischemia-injured muscles of the posterior part of the thigh, which are required to convey and to deliver blood to tissues. Induction of such mature vessels represent a particularly efficient treatment in most severe cases where adverse angiogenesis defects are elicited by hypercholesterolemia or diabetes.

Outgrowth of both collaterals and arterioles is of particular interest in therapeutic angiogenesis. Indeed, collaterals are vessels forming bridges between arterial networks while arterioles are mature vessels formed of a layer of endothelial cells stabilized by mural cells (pericytes or smooth muscle cells) providing bulk flow to the tissue. Capillary networks are therefore dependent on their presence for ensuring distribution of the flow. (Carmeliet et al., Nat. Med., 2000; 6:389-395; Van Royen et al., Cardiovasc. Res., 2001;49:543-553).

A further object of the present invention is to provide a method of promoting angiogenesis with the provisio that VEGF is not upregulated in the treated cells.

The following examples demonstrate that intramuscular administration of injection of NV1FGF does not lead to local murine VEGF-A induction in injected muscles and does not lead to murine VEGF-A secretion in the circulating blood of the injected mice. This is an important aspect of the present invention, which is related to a new method for promoting collateral blood vessels and arterioles, without inducing the VEGF-A factor, in a VEGF-independent pathway. In effect, it is well known that VEGF-A cause serious negative side effects such as promiscuous unwanted angiogenesis, edema, and potential of tumorigenicity.

Throughout this application, various publications, patents and patent applications have been referred to. The teaching and disclosures of these publications, patents and patent applications in their entireties are hereby incorporated by reference into this application to more fully describe the state of the art to which the present application pertains.

It is also understood and expected that variations in the principles of invention herein disclosed in an exemplary embodiment may be made by one skilled in the art and it is intended that such modifications, changes and substitutions are to be included within the scope of the present application.

### EXAMPLES

### Example 1

### Animals and Diets

Syrian Golden hamsters (n=50) of 11-12 weeks (CERJ, Le Genest St Isle, France) are used in the experi approved by the Animal Use Committee and conducted in accordance with guidelines published by the National Institute of Health (NIH publication No. 85-23, revised 1985).

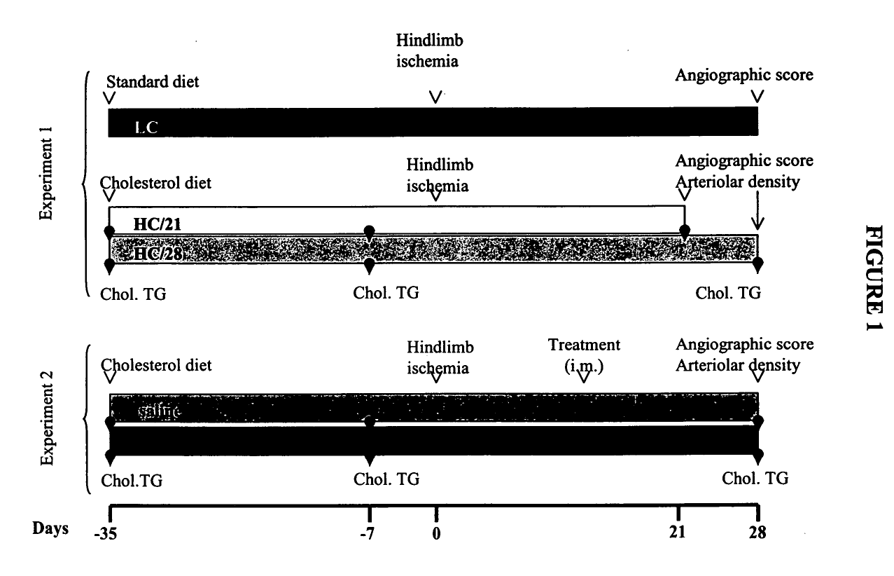

In experiment 1, hamsters (n=37) are randomly divided into three groups (FIG. 1). Hamsters in the low cholesterol (LC) group (n=13) are fed ad libitum with standard chow (ref. A04-C, UAR, Epinay-sur-Orge, France). Hamsters in both high cholesterol (HC) groups during 21 days (HC/21) and 28 days (HC/28) post-treatment (HC/21: n=12; HC/28: n=12) are given 20 g per animal of cholesterol-enriched diet daily, made of standard chow supplemented with 3% cholesterol and 15% cocoa butter (ref. 1414C, UAR, Epinay-sur-Orge, France). In experiment 2, hamsters (n=13) are randomly allocated to two groups (FIG. 1). Hamsters in the saline group (n=5) and in the NV1FGF group (n=8) are fed with cholesterol-enriched diet, as described in experiment 1.

### Example 2

### Induction of Hindlimb Ischemia

After 35 days of LC or HC diet, animals are subjected to hindlimb ischemia, according to the following surgical procedure. Hindlimb ischemia is induced under gas anesthesia with N₂O (0.8 l.min⁻¹), O₂ (0.4 l.min⁻¹) and isofluorane (2%) according to a procedure described in other animal species (19,20). Under sterile surgical conditions, a longitudinal incision is performed on the medial thigh of the right hindlimb from the inguinal ligament to a point proximal to the patella. Through this incision, using surgical loops, the femoral artery is dissected free and its major branches coagulated. The femoral artery is completely excised from its proximal origin as a branch of the external iliac artery to the point distal where it bifurcates into the saphenous and popliteal branches (20). The incision is closed in one layer with a 4.0 silk wire.

### Example 3

### Gene Transfer in Hindlimb Skeletal Muscles

In experiment 2, saline (n=5) or plasmid encoding NV1FGF (180 μg DNA, n=8) is given blind 14 days after induction of ischemia, through three 50-μL injections each in Tibialis cranialis, Adductores and Quadriceps muscles of the ischemic limb.

### Example 4

### Measurements of Total Cholesterol and Triglyceride Levels in Serum

On days −35, −7, and +21 or +28 related to the day of surgery, blood is obtained from hamsters of HC/21, HC/28, saline and NV1FGF groups by retro-orbital puncture under gas anesthesia with N₂O (0.8 l.min⁻¹), O₂ (0.4 l.min⁻¹) and isofluorane (2%). Total cholesterol and triglyceride levels in serum are determined enzymatically with commercially available kits (Olympus Diagnostica GmbH, Hamburg, Germany).

### Example 5

### Quantification of Collateral Vessel Formation by Angiography

On day 21 (HC/21 group) or day 28 (LC, HC/28, saline and NV1FGF groups) after induction of ischemia, an angiographic procedure is performed as follows.

Immediately after injection of ˜300 μl of contrast medium (0.5 g.ml⁻¹ sulfate barium solution in water) through a catheter inserted into abdominal aorta, hamsters are sacrificed with an overdose of sodium pentobarbital. Hamsters are placed in dorsal decubitus into a radiography apparatus (model MX-20, Faxitron X-ray Corp., Wheeling, Ill., USA) and post-mortem pictures of the vasculature from both limbs collected and digitalized (software Specimen, DALSA MedOptics, Tucson, Ariz., USA). This radiographic system allows visualization of vessels with diameters higher than 150 μm. Pictures are analysed off-line by an investigator blinded to the treatment, with dedicated software as previously described (22).

Briefly, for both ischemic and non-ischemic limbs, the extent of collateral vessels in the posterior side of the thigh is determined as a percentage of the area analysed. Angiographic score is calculated as the ratio ischemic/non-ischemic percentages. In order to check the validity of the method, angiographic score is assessed in six separate age-matched hamsters not subjected to hindlimb ischemia. As expected, angiographic score calculated as the ratio right limb/left limb percentages was 1.04±0.18, reflecting similar vascularization in both limbs.

### Example 6

### Quantification of Arteriolar Formation by Immunohistochemistry and Typical Muscle Lesions Induced by Hindlimb Ischemia Through Excision of the Femoral Artery of Hypercholesterolemic Hamsters

On day 21 (HC/21 group) or day 28 (LC, HC/28, saline and NV1FGF groups) after induction of ischemia, skeletal muscles from the ischemic hindlimb are harvested and fixed in a solution of PBS-3.7% formaline. Muscles from the non-ischemic hindlimb are sampled similarly and served as control muscles. Two transverse slices composed of different muscles (Gracilis, Semimembranosus, Adductores, Semitendinosus, Biceps femoris), are processed from the back part of each thigh. Slices are dehydrated, embedded in paraffin and 5-μm thick sections are prepared for immunohistochemistry. A mouse monoclonal antibody directed against smooth muscle α-actin (SMA; clone 1A4, dilution 1:200, Dako, Carpinteria, Calif., USA) is used as a marker for vascular smooth muscle cells (VSMCs) since it is constitutively expressed in mature vessels. The SMA antibody is detected with a commercially available kit (Envision™+System/Horse Radish Peroxidase, Dako, Carpinteria, Calif., USA) through an avidin-biotin-peroxidase method. SMA-positive (SMA+) vessels are ranked by size (outer diameter) and arterioles with diameter <50 μm were counted in both Adductores and Gracilis muscles.

These muscles have been chosen for their susceptibility to histopathological lesions in our hindlimb ischemia hamster model, conversely to other muscles from the posterior side of the thigh (Semitendinosus, Semimembranosus and Biceps femoris).

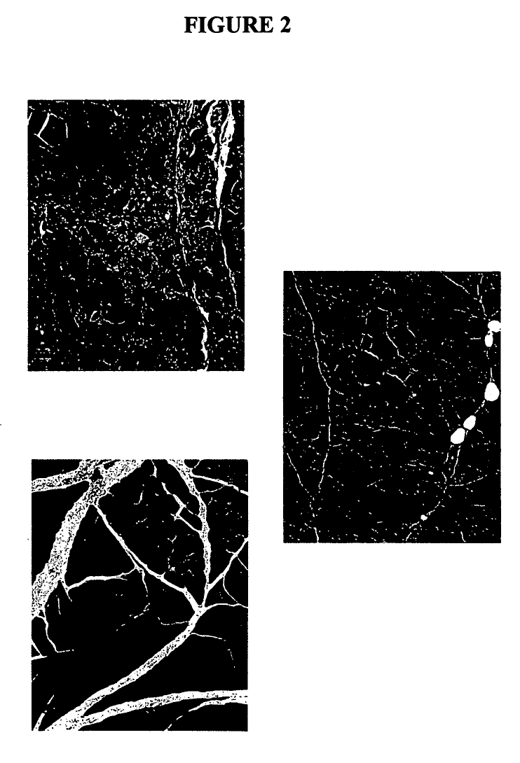

Typical lesions induced by excision of femoral artery are shown in FIG. 2. Muscles from the back part of the thigh, i.e., Gracilis and Adductores are harvested 28 days after induction of ischemia, and 5 μm thick sections of the muscles after HES staining are observed. FIGS. 2B and 2C show the presence of mild necrosis (dashed line) and centronucleation (arrows) in ischemic muscles, respectively. FIG. 2A shows a cross-section at magnification ×100 of the non-ischemic controlateral muscles having no lesions, as a control. Total area of Adductores and Gracilis muscles was determined to investigate the impact of ischemia on muscle volume. Number of SMA+ arterioles was determined for the total muscle area. For both parameters, the ratio ischemic/non-ischemic values was then calculated. All procedures were performed by an investigator blinded to the treatment.

### Example 7

### Expression of FGF-1 after NV1FGF Gene Transfer in Ischemic Muscles

In experiment 2, 14 days after saline injection or NV1FGF gene transfer (i.e., 28 days after induction of ischemia), muscles from the back part of the thigh (Gracilis and Adductores) from non-ischemic and ischemic limbs are processed as follows. FGF-1 immunohistochemistry is performed using a classical streptavidin-biotin assay used to detect FGF1 expression. The incubation with a primary polyclonal anti-FGF-1 rabbit antibody (reference AB-32-NA, 1:30 dilution, R&D Systems, Abingdon, UK) is followed by incubation with a biotinilated donkey anti-rabbit immunoglobulin (1:200 dilution, Amersham, Buckinghamshire, UK). The immune complexes are localized using a chromogenic diaminobenzidine substrate, after adding peroxidase coupled to streptavidine. The 5-μm thick sections are counterstained with hematoxylin, dehydrated and mounted with permanent mounting media. Immunoreactive fibers are identified (brown staining) under a microscope (Axioplan 2, Zeiss, Hallbergmoos, Germany).

Statistical Analysis

Results are expressed as mean SD. Statistical significance was assumed at P<0.05.

In experiment 1, serum lipid levels in groups HC/21 and HC/28 were compared at the various timepoints by ANOVA followed by Tukey-Kramer post-test for comparison between any 2 values. Angiographic scores calculated in groups LC, HC/21 and HC/28 were compared by ANOVA followed by Tukey-Kramer post-test. Non-ischemic and ischemic values of muscle area and number of SMA positive arterioles, as well as corresponding ratios, were compared by unpaired t-test in groups HC/21 and HC/28.

In experiment 2, serum lipid levels in saline and NV1FGF groups were compared at the various timepoints by ANOVA followed by Tukey-Kramer post-test for comparison between any 2 values. Angiographic scores, non-ischemic and ischemic values of muscle area and number of SMA positive arterioles, as well as corresponding ratios, were compared by unpaired t-test in saline and NV1FGF groups.

### Example 8

### Measure of Serum Lipids in Low Cholesterol and Cholesterol-Rich Diet

Tables 1 and 2 summarize serum lipid levels in experiment 1 and experiment 2, respectively, before cholesterol-rich diet was given (day −35) and at the various timepoints following diet modification (days −7 and +21 or +28). Cholesterol-rich diet led to a time-dependent increase both in total cholesterol and triglyceride serum levels.

TABLE 1
Serum lipids in experiment 1

| | HC/21 | HC/28 |
|---|---|---|
| Total cholesterol (mg · dl⁻¹) | | |
| Before diet | 157 ± 37 | 144 ± 43 |
| D-7 | 1118 ± 384*** | 859 ± 182*** |
| D21 | 1910 ± 393*** | N.A. |
| D28 | N.A. | 2016 ± 455*** |
| Triglycerides (mg · dl⁻¹) | | |
| Before diet | 173 ± 42 | 226 ± 56 |
| D-7 | 263 ± 79 | 324 ± 258 |
| D21 | 1644 ± 835*** | N.A. |
| D28 | N.A. | 1841 ± 1083*** |

N.A.: non applicable
***P < 0.001 vs. before diet

TABLE 2
Serum lipids in experiment 2

| | Saline | NV1FGF |
|---|---|---|
| Total cholesterol (mg · dl⁻¹) | | |
| Before diet | 167 ± 43 | 165 ± 16 |
| D-7 | 1468 ± 304*** | 1359 ± 413*** |
| D28 | 1829 ± 267*** | 1876 ± 537*** |
| Triglycerides (mg · dl⁻¹) | | |
| Before diet | 154 ± 30 | 128 ± 27 |
| D-7 | 617 ± 253** | 517 ± 233*** |
| D28 | 779 ± 681** | 703 ± 280*** |

N.A.: non applicable
**P < 0.01 vs. before diet
***P < 0.001 vs. before diet

### Example 9

### Effect of Cholesterol-Rich Diet on Collateral Development and Arteriolar Density After Hindlimb Ischemia (Experiment 1)

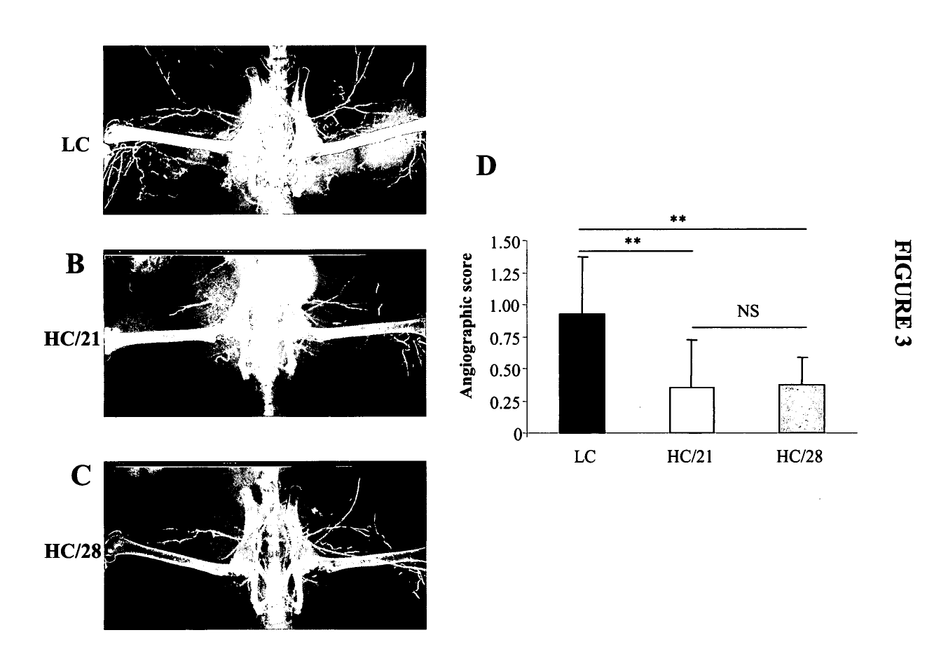

As illustrated in FIGS. 3, collateral formation 28 days after hindlimb ischemia was high in LC group (FIG. 3A), leading to angiographic score of 0.93±0.45 (FIG. 3D).

Conversely, in both HC groups formation of collaterals was dramatically impaired 21 or 28 days after hindlimb ischemia with angiographic scores decreased to 0.35±0.38 and 0.37±0.21, respectively (see FIGS. 3B and 3C). Decrease in angiographic score was significant in both HC groups compared to LC group (P<0.01). Nevertheless, angiographic scores were not different between HC/21 and HC/28 groups (P>0.05) as illustrated in FIG. 3D.

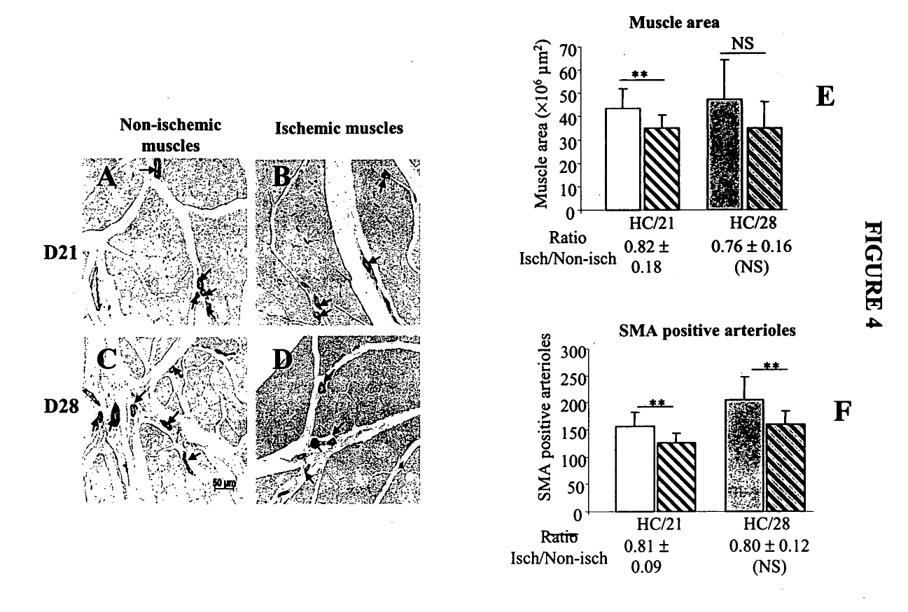

FIGS. 4A-4D display representative cross-section at magnification ×100, depicting mature vessels labeled by smooth muscle α-actin (SMA) immunohistochemistry from non-ischemic and ischemic muscles (Adductores and Gracilis) harvested at day 21 or day 28 after induction of ischemia and quantification of muscle area and <50 μm SMA− positive arterioles.

As illustrated in FIGS. 4E-F, area of Adductores and Gracilis muscles decreased in the ischemic limb compared to the non-ischemic limb, 21 days after ischemia (P<0.01). Though failing to reach statistical significance (P=0.0538), the same trend was observed 28 days after hindlimb ischemia. In addition, the number of <50 μm arterioles significantly decreased in the ischemic limb compared to the non-ischemic limb (P<0.01), both 21 and 28 days after ischemia. Despite these statistical levels of significance in the intermediate steps of calculation of arteriolar density, density itself was not different 21 and 28 days after initiation of diet modification. Muscle area, SMA+ arterioles and arteriolar density expressed as ratios ischemic/non-ischemic limb were not different on days 21 and 28.

These results clearly demonstrate that hypercholesterolemia induces an inhibition of the development of collateral vessels, and a chronic angiogenic disorder both at the macro- and microvascular levels, as quantified by angiography, and of <50 μm arterioles, quantified by histological methods to the same extend, 21 and 28 days after hindlimb ischemia (FIGS. 2B and 2C).

In addition, the hypercholesterolemic hamsters used provide a particularly severe model, as the lipid overload applied to our model is elevated and clearly results in endothelial dysfunction and defect in angiogenesis response after hindlimb ischemia. Histopathological analysis of arteries harvested from hamsters 4 weeks after initiation of the cholesterol-rich diet revealed the presence of foam cells. Furthermore, there is a continuous increase in the total cholesterol and triglycerides levels lasting during the period of recovery from hindlimb ischemia, so that the model resembles the worst case scenario in terms of severity of the endothelial impairment and angiogenesis defects.

### Example 10

### Effect of NV1FGF Gene Transfer on Collateral Development and Arteriolar Density 14 Days After Intramuscular Administration (i.e., 28 Days After Hindlimb Ischemia) in Hypercholesterolemic Hamsters (Experiment 2)

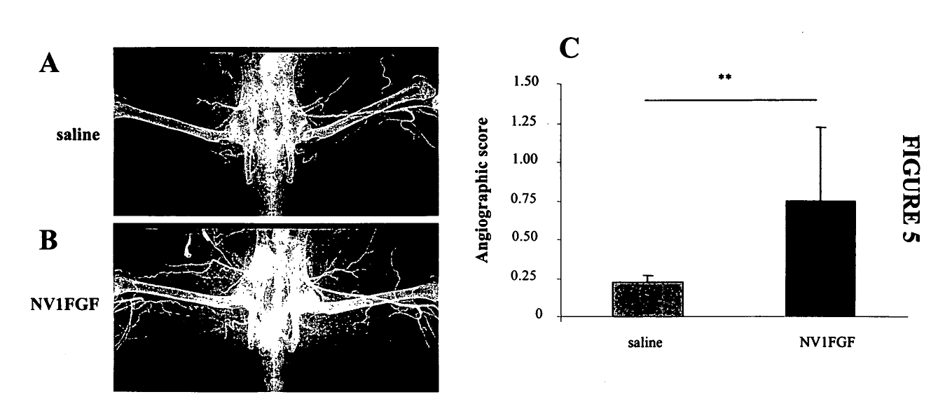

As indicated in FIG. 5B, intramuscular NV1FGF gene transfer 14 days after induction of hindlimb ischemia greatly improved collateral formation in the ischemic limb, when compared with saline-treated hamsters (FIG. 5A). Also, angiographic score after NV1FGF gene transfer (0.75±0.47) was indeed significantly higher (P<0.01) than that of saline-treated hamsters (0.22±0.05).

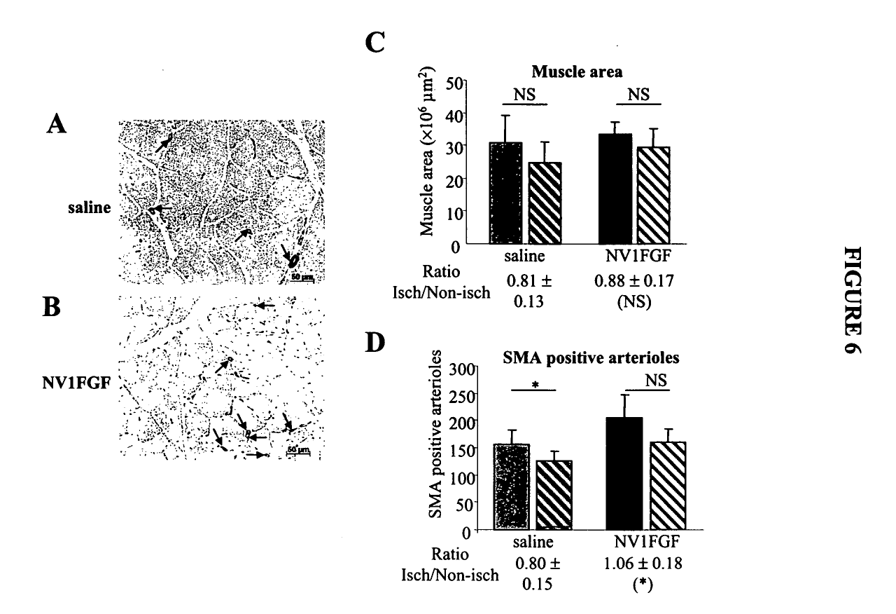

FIGS. 6A and 6B which are representative cross-sections (magnification ×100) depicting mature vessels labeled by smooth muscle α-actin (SMA) immunohistochemistry from ischemic muscles of hamsters treated with saline and NV1FGF and quantification of muscle area and <50 μm SMA positive arterioles.

A decreased area of Adductores and Gracilis muscles in the ischemic limb was observed in both saline and NV1FGF groups (P=0.2404 and P-0.0846, respectively). As indicated in FIGS. 6C and 6D, muscle area expressed as ratio ischemic/non-ischemic limb was similar in saline and NV1FGF groups (P=0.4584). The number of <50 μm arterioles decreased significantly in the ischemic limb compared to the non-ischemic limb (P=0.0333) in saline-treated animals, whereas the difference was not significant in NV1FGF group (P=0.1347). Calculation of the ratio ischemic/non-ischemic limb revealed a statistically higher value in NV1FGF-treated muscles compared to saline (P=0.0187). Nevertheless, arteriolar density was not different in muscles from non- ischemic and ischemic limbs of either treatment group (P=0.9320 and P=0.1586 for saline and NV1FGF, respectively). Arteriolar density expressed as the ratio ischemic/non-ischemic limb was not different between saline and NV1FGF groups (P=0.2724).

### Example 11

### Expression of FGF-1 in Ischemic Muscles After NV1FGF Gene Transfer

In contrast with gene therapy involving other angiogenic factors, such as VEGF or FGF-2, the inventors have evidenced that the expression of FGF-1 was advantageously restricted to the ischemic muscles of animals treated with NV1FGF.

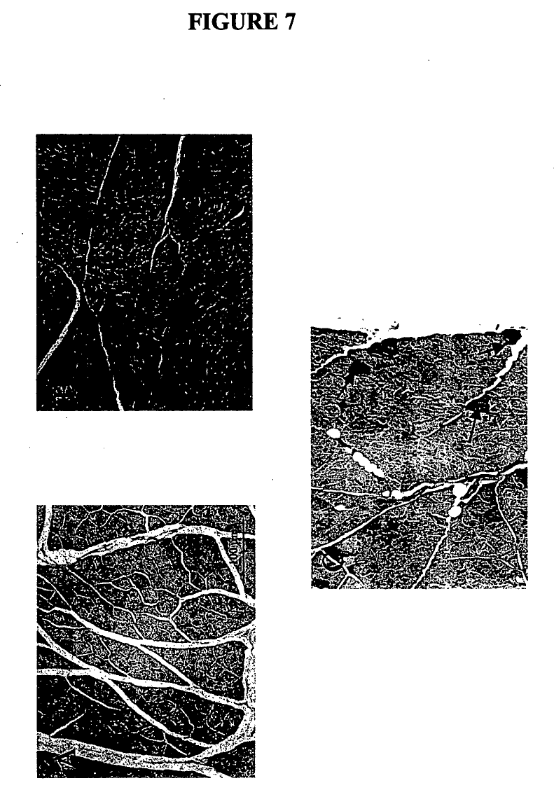

Also, as shown by representative pictures (magnification ×100) of immunohistochemical staining with an anti-FGF-1 polyclonal antibody in muscles from the back part of the thigh (Gracilis and Adductores) from non-ischemic (controlateral) non injected limbs (FIG. 7A) and ischemic limbs injected with saline (FIG. 7B) or with NV1FGF (in FIG. 7C), the expression of FGF-1 could surprisingly be detected neither in the ischemic muscles of saline-treated animals, nor in non-ischemic (controlateral) muscles of saline and NV1FGF-treated animals. This clearly shows the superior properties of the plasmid NV1FGF which allows a slow release of the encoded FGF-1 protein within a therapeutic window sufficient to effect a sustained angiogenic response via the formation of mature blood vessels, but at a concentration which does not permit dissemination and promiscuous angiogenesis or negative side effects. NV1FGF was thus proved to be particularly potent, as being capable of efficiently promoting angiogenesis at a non-detectable concentration in treated muscles, thus allowing use of concentrations of NV1FGF comprised within a therapeutic window and in conditions characterized by aggravated endothelial dysfunctions. Due to such superior characteristics in terms of safety and potency, the NV1FGF may advantageously be used as angiogenesis therapy in aggravated conditions caused by hypercholesterolemia or diabetes.

These results clearly demonstrate that NV1FGF gene therapy is capable of rescuing impaired tissue by an increase of collateral vessels and arterioles. In effect, the growth of >150 μm collateral vessels has been evidenced angiographically and the growth of <50 μm arterioles as evidenced by immunohistochemistry in the posterior part of the thigh, which comprises Biceps femoris, Adductores, Gracilis, Semimembranosus, and Semitendinosus muscles. Unexpectedly, the Applicant has demonstrated that formation of collateral vessels was significantly stimulated into this region, 14 days after NV1FGF gene transfer, as emphasized by angiographic score (FIG. 5C).

Though no in situ measurements of tissue oxygenation has been performed to assert that ischemia occurred in that region after femoral artery excision, the presence of histological lesions such as centronucleation, dystrophy, necrosis and inflammation was revealed (FIG. 2). More precisely, these lesions were restricted to Adductores and Gracilis muscles whereas Biceps femoris, Semimembranosus, and Semitendinosus muscles were not prone to lesions. Quantification of <50 μm arterioles was therefore performed exclusively in Adductores and Gracilis muscles, just above the adductor canal. As demonstrated in FIG. 6D, NV1FGF gene transfer increased the absolute number of arterioles into these muscles of the ischemic limb.

These data thus unambiguously demonstrate the ability of NV1FGF to induce the formation of mature large conductance vessels (>150 μm collateral vessels) and small resistance arteries (<50 μm arterioles) in ischemia-injured muscles of the posterior part of the thigh, which are required to convey and to deliver blood to tissues.

### Example 12

### Absence of VEGF-A Induction in NV1FGF Treated Muscles

The purpose of this experiment is to assess in vivo VEGF-A induction following intramuscular (IM) administration of naked DNA encoding rat FGF-1 in mice. Both murine VEGF-A (mVEGF-A) circulating levels and local gene expression are investigated. The circulating levels are measured by ELISA 3 and 7 days following IM dosing, and local mVEGF-A gene expression investigated by day 7 using two assays: immunohistochemistry (IHC) against the protein and detection of mRNA by Real-Time Reverse Transcription Polymerase Chain Reaction (RT-PCR).

Forty female mice are used in this study. The groups 1 to 3 (n=8 per group) receive IM administrations of pCOR-CMV.rat-spFGF-1, pCOR-CMV.Empty (without FGF-1 gene) or NaCl 0.9% respectively, in both right and left Tibialis Cranialis muscles. The pCOR-CMV.rat-spFGF-1 corresponds to the NV1FGF plasmid wherein the human FGF-1 was replaced by the corresponding rat-derived coding sequence. The injected muscles are harvested on day 7 following dosing and processed for mVEGF-A and FGF- 1 immunohistochemistry (right muscles) or mVEGF-A Real-Time RT-PCR (left muscles). In groups 1 to 3, blood is collected on day 3 (D3) and day 7 (D7) post-dosing for ELISA detection of mVEGF-A in freshly prepared serum samples.

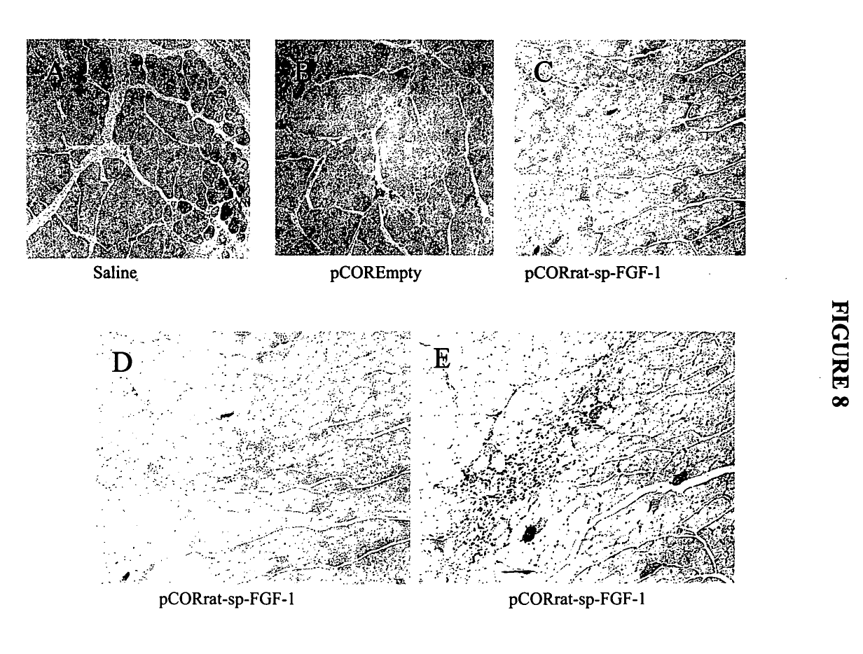

As shown in FIG. 8, no FGF-1 positive myofibers were detected in pCOR- CMV.Empty I (FIG. 8B) nor NaCl injected muscles (FIG. 8C). A mean of 25 FGF-1 expressing myofibers/section was established for pCOR-CMV.sp-ratFGF-1 injected muscles (FIG. 8E).

Using immunohistochemistry, a similar and faint labeling of mVEGF-A was observed in both groups of control muscles (NaCl and pCOR-CMV.Empty), indicating endogenous expression of mVEGF-A. The immunostaining, localized within the myofibers, displayed a mosaic pattern, as some fibers were more intensely labeled than others. In muscles injected with pCOR-CMV.rat-spFGF-1, no increase of mVEGF-A immunolabeling was observed by D7 post-dosing (FIG. 8D) when compared to pCOR-CMV.Empty or NaCl injected muscles. The same mosaic pattern of immunostaining was observed.

Using Real-Time RT-PCR technology, similar levels of mVEGF-A mRNA expression were evidenced in pCOR-CMV.Empty and NaCl treated muscles (6.84×10³ and 4.31×10³ cDNA copies per 2 ng of total RNA), indicating endogenous mVEGF-A expression. In pCOR-CMV.sp-ratFGF-1 injected muscles, no increase in mVEGF-A mRNA level (6.51 10³ cDNA copies per 2 ng of total RNA) was observed by D7 post- dosing, when compared to pCOR-CMV.Empty or NaCl injected muscles.

These results clearly indicate that intramuscular administration of pCOR- CMV.rat-spFGF-1 did not lead to local mVEGF-A induction in injected muscles and did not lead to mVEGF-A secretion in the circulating blood of the injected mice.

### Example 13

### Hypercholesterolemic Watanabe Rabbit Coronary Artery Disease Animal Model

The validation of a gene therapy product as a potential treatment partly stands on its biological activity. In order to assess it, the animal model of choice should be as close as possible to the human disease it mimics as far as anatomy and function are concerned.

The anatomical relevance was based on the species used, which should be as close as possible to the human heart, as far as the coronary network is concerned. Moreover, the bigger species the better, as it eased the various technical steps. On the other hand, factors like the cost, handling facility, animal status contingency, and animal facility compliance was addressed. The best compromise found in the present study is the rabbit, small enough to be easily handled and immobilized, and big enough to allow a good spotting of a particular artery, a precise coronarography, and a human-like comparison of various anatomical and functional issues.

Indeed, the functional relevance was here based on the ability of the animal to develop pathology as close as possible to its human counterpart, i.e. angiogenic defects associated with hypercholesterolemia.

Hypercholesterolemia in humans causes a vascular endothelial dysfunction and ultimately a progressive narrowing of the main coronary arteries. The unbalance in the coronary blood flow at rest and during stress creates a furtive malfunction of the myocardium that leads to pain and hypocontractility. Usually the supplies are appropriate at rest, but when stress occurs; the needs increase while the supplies cannot, due to the coronary obstructive lesions. This is the reason why the purpose of mimicking this human pathology, leads to both the setup of a stenosis on a major coronary artery and the use of a stress test to reveal the unbalance created at stress by this stenosis.

Watanabe Heritable Hyperlipidemic Rabbits (WHHR) which lack of LDL receptor, therefore developing a spontaneous atherosclerosis that leads to coronary atheroma were thus used to assess ischemia and evaluate the effect on their ischemic status of intramyocardial injections of NV1FGF during open chest surgery.

All experiments were conducted in accordance with a protocol approved by the Animal Care and Use Committee and conform to the NIH Guidelines for the Use and Care of Laboratory Animals.

WHH one-year-old rabbits weighing 3000 to 3500 g were obtained from Covance (PO Box 7200, Denver, Pa., 17517, USA).

All the animals were kept in animal quarters according to good animal care practices for at least eight days preceding their utilization. Throughout this period, they were housed one per cage, had free access to food (112C type from UAR) and appropriately filtered drinking water. The animal house was maintained on a 12-h light/dark cycle (lights on at 6 a.m.) with an ambient temperature of 20-24° C. and humidity set at 35-75%.

Watanabe rabbits had cholesterol levels 7 to 10 times higher than the wild type animals, while their triglycerides levels were 4 to 5 times higher than normal.

After sacrifice, four of them underwent an Oil Red'O staining which evidences the presence of lipid plaques in the aorta, the coronary ostia and the mitral valves.

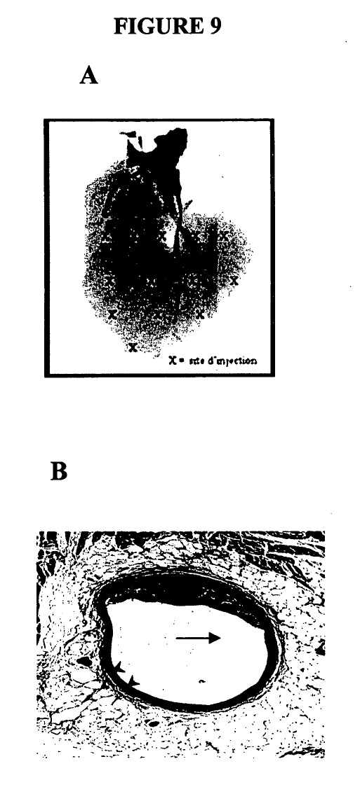

Furthermore, one of those was submitted to histological analysis. Microscopic examination of HES stained sections of the circumflex coronary artery identified an atherosclerotic plaque covering about of 15% of the lumen in one of the two studied rabbit (Rabbit #CAD98R2, see FIG. 9B).

The Watanabe Heritable Hyperlipidemic Rabbit possessed two interesting properties: the size of its heart was well adapted to multiple intramyocardial injections, and its coronary network was studded with atherosclerotic plaques. The latest explained why the coronary blood flow is normal at rest (as assessed by a normal ECG and a normal contractility), while a dobutamin stress test under anesthesia lead to a marked pattern of ischemia.

In fact, both analysis, e.g., electrocardiography and echocardiography, evidences at stress signs that can be compared to their human counterparts, for example ST depression or hypokinesis.

### Example 14

### Surgery

Surgery is used to deliver the gene therapy product into the myocardium by direct injection. The surgical procedure is performed under sterile conditions. The animals are anesthetized with an intramuscular injection (1 ml/Kg) of a mixture of ketamine (70 mg/Kg)+Xylazine (7 mg/Kg). After the animals are shaved, they are vaporized with Lidocaine 5% spray in the throat to make easier the endotracheal intubation, and the anesthesia is maintained throughout the experiment with a mechanical ventilator (Siemens, Servo Ventilator 900D) with the following conditions:
Insufflation volume: 1.4 L/min
Breathing frequency: 40 /min
Halothane: 0.4 to 0.6%
Oxygen: 30-35%

A cannulation of the marginal ear vein is done so that the animal is continuously perfused with 5% of glucose. A monitoring by ECG is used to ensure a stable rhythm throughout the surgical act. A left thoracotomy is performed on the fourth intercostal space. After the opening of the pericardium, the heart is exposed for plasmid injection.

The injections are made on 13 different locations on the free wall of the left ventricle with a compositve device: 250 μl Hamilton syringe connected to a Stëriflex G19 catheter (ref: 167.10) fixed with a short bevel needle BD (26G^{3/8}). We injecte 25 μl per location of a 1 mg/ml solution of plasmid. FIG. 1A shows the location of each injection.

After the injections, a drainage tube is placed in the thoracic cavity and the ribs are put side by side with two Mersurtures® (1.0) threads. The Halothane is stopped and oxygen maintained until the wakening of the animal. The drainage is set around 200-400 mbar throughout the closing of the thorax. Two layers of muscles are closed with a Vicryl® (4.0) thread. The skin is then stitched with a Suturamide (2/0) thread. When the last stitch is done and before removal of the draining tube, the end expiration positive pressure (PEEP) is increased in the lungs with the help of the mechanical ventilator so that the lungs are well inflated throughout the thorax just before the draining tube is pulled out.

A betadine gel is applied and a bandage is dressed on the wound. When the animal wakes up the mechanical ventilator is stopped.

### Example 15

### Post-Surgery Treatment

As soon as the animal is awake, the following compounds are injected:
an anti-aggregate and an anti-coagulate to avoid potential thrombosis of the coronary artery during and after the surgery:
   0.375 ml of Vetalgine (one day before surgery and then during 5 days): intramuscular
   0.1 ml heparin (for 4 days from next day of surgery): subcutaneous
an antibiotic with a large spectrum to prevent any infections:
   0.2 ml of Baytril 2% (for 5 days): subcutaneous
a strong analgesic so that the animal withstands the heavy surgical act:
   0.5 ml of morphine (for 2 days): subcutaneous

### Example 16

### Electrocardiography

Electrocardiography is set up as close as possible to its human counterpart. Four electrodes are set on the four limbs, and six (V1 to V6) are set on the precordium. A classical 12 lead ECG is recorded on a HP Pagewriter II 4565A. The use of the ECG follows enables one to monitor: (1) the cardiac rhythm during the surgery; (2) the cardiac rhythm during dobutamin stress test; (3) the detection of myocardial infarction; and (4) the detection of the signs of ischemia.

4.1 Monitoring of Cardiac Rhythm During the Surgery

An open chest surgery with total anesthesia could potentially lead to various per- operatory problems that can be detected by ECG monitoring. Bradycardia is treated by atropin injection, arythmia by Lidocaine 0.5% injection (0.5 to 1 ml), and cardiac arrest by energic cardiopulmonary resuscitation including Isoprenaline.

4.2 Monitoring of the Cardiac Rhythm During the Dobutamin Stress Test

Even if the main pharmacological effect of dobutamin was its action on inotropy, this product also seemed chronotropic, and the increase in cardiac rhythm thus appeared the easiest way to monitor the stress test.

4.3 Detection of Myocardial Infarction

Cardiac electric activity was recorded as a series of beats, each of them being a succession of waves: p, q, r, s and t, for the most important part. The p wave was used as a witness of the atrial activity, the qrs complex the ventricular activity, and the t wave was used as a marker of repolarization.

The most classical ECG sign of myocardial infarction in humans is a deep and large q wave. The parallel could be done with rabbits, as the analysis of an exploratory series of rabbits showed the presence of such a q wave in most of them when they underwent a mechanical closure of a coronary artery.

4.4 Detection of Signs of Ischemia

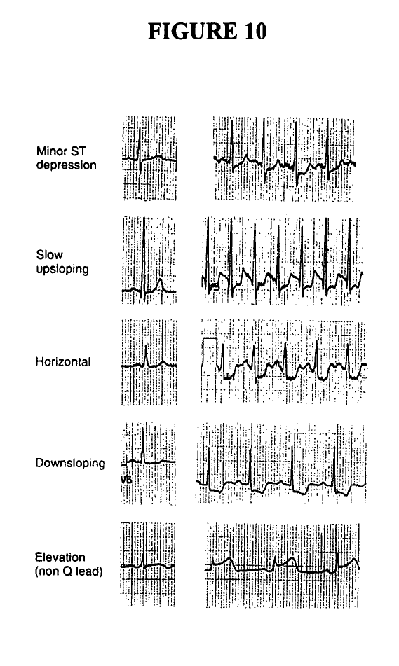

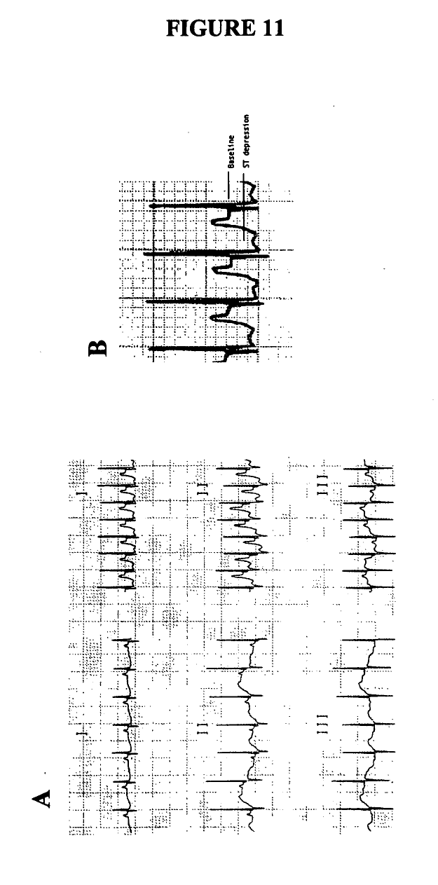

This furtive phenomenon is usually not seen at rest. During a stress test various modifications are described. We took the assumption that a rabbit heart showed the same electrical pattern when it undergoes the same mechanical/chemical stress. In fact, a real ischemia in humans leads to an ST-segment elevation or depression, or/and inversion of the T wave (FIG. 10). The two main signs are the ST-segment depression and the negative deep T wave, as shown in FIG. 10 during a human stress test. An example of its rabbit counterpart was showed in FIGS. 11.

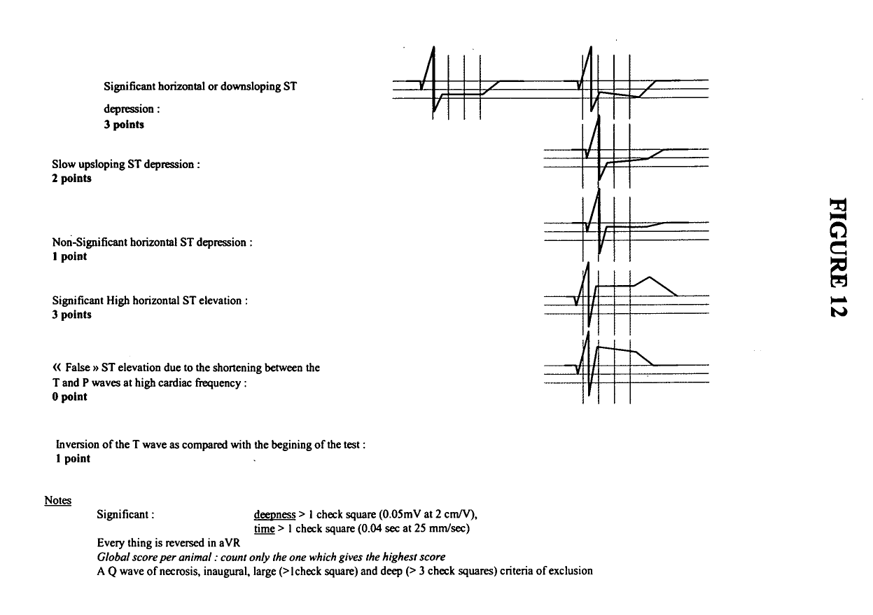

The method of scoring signs of ischemia was shown in FIG. 12. A score from 0 (normal ECG) to 3 points (significant ischemia) for each lead as shown in FIG. 12 was given and recorded. The presence of a significant deviation in any lead was as important as the number of leads where the deviation was found. Therefore, at first the sum of all scores was recorded, then only the highest score found on a particular ECG was kept and used for the inclusion of ischemic animals. The only exclusion criteria was the presence of a q wave (larger than 1 square and deeper than 3 squares) indicating a transmural necrosis.

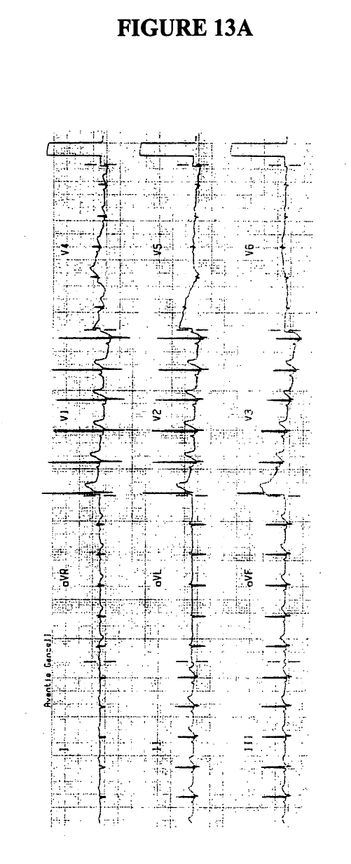

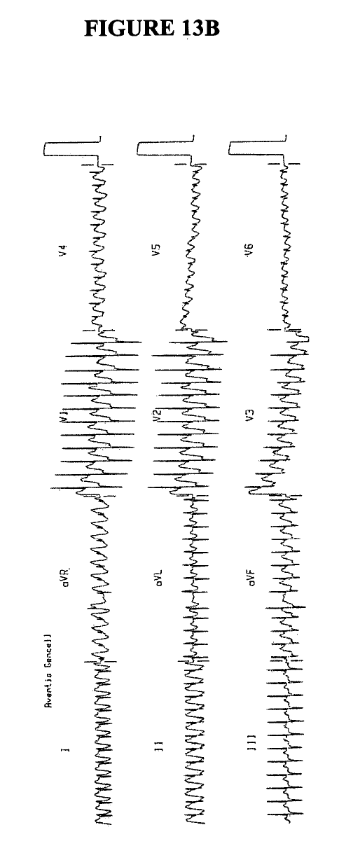

A typical normal 12 lead ECG at rest was displayed in FIG. 13A, while the same animal at maximum stress (see FIG. 13B) showed a significant downsloping depression in lead I, II, aVF, V1, V2, V3, V4, and a non-significant depression in lead II1, V5 and V6. This particular ECG showed a very strong ischemia.

In conclusion, the ECG was the best first line method to detect either an exclusion criteria (i.e. necrosis), or an inclusion criteria (ischemic response to dobutamin).

### Example 17

### Echocardiography

An Acuson Sequoia 256 and a linear 8L5 8 MHz probe is used to assess the myocardium contractility, as the small size of the rabbit thoracic area allowed using this superficial probe in this particular analysis.

For each animal, once before the surgery, then every week before and during the stress test, an echocardiography is performed. In each case, the overall kinetic behavior of the heart is examined, in as many segments as possible one after the other, in both long axis and short axis. Then in long axis the largest diameter is spotted, and the apparatus switched into M mode to ensure a normal behavior of the left ventricle.

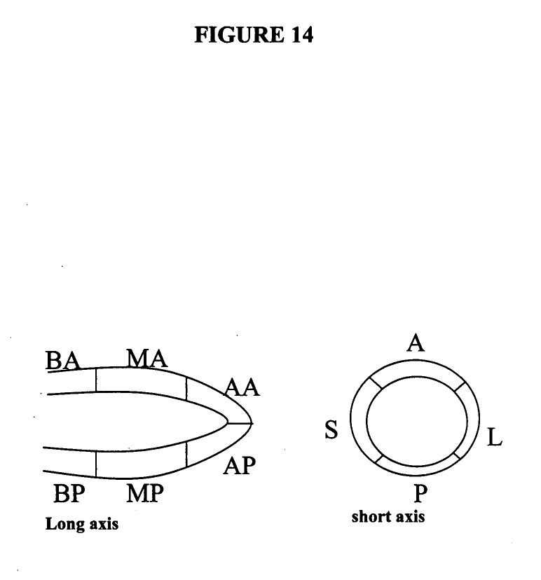

A similar 2D analysis is performed at each step of the stress test. Any detected abnormality is recorded and further evaluated: the location is noted by using the nomenclature below (FIG. 14), and the type of defect is rated: 1 for normal, 2 for hypokinesy, 3 for akinesy, 4 for dyskinesy. When there is a doubt on the presence of a defect, the thickening fraction is rated using M mode. A sequence is thereafter scored as 1 if all the segments have a normal thickening fraction (above 30%), 2 if hypokinetic (one or more segments under 30%), 3 if akinetic (at least one segment with no thickening), or 4 if dyskinetic (one segment with negative thickening fraction, i.e. the myocardium expends during the systole).

Only the highest defect score is kept for the final analysis.

### Example 18

### Dobutamin Stress Test

This test used dobutamin for its inotropic and chronotropic properties in order to mimic the cardiac response to stress conditions. As ischemia is a furtive phenomenon that usually occurs during a stress, its unveiling is detected by an electric signature on the ECG and its consequences on myocardial contractility, as evidenced by echocardiography. The technical steps of the test are as follows.
Anesthesia: Isoflurane was discarded as it leads to high heart rate at rest. Halothane was discarded as the addition of atropin lead to the appearance of ventricular ectopies. Xylazine-Ketamine was therefore chosen, as the kinetics of the heart rate increase was good.
Atropin: 0.5 mg/Kg of atropin methyl nitrate were injected in the ear catheter as a preliminary step. Atropin sulfate was discarded at it lead to some degree of heart rate increase.
Scale up of the doses: The first dose was 2 μg/Kg/min. Every three minutes the dose was increased to 5, 10, 20, 30, and 40 μg/kg/min. If the maximal heart was not reached, a final dose of 80 μg/Kg/min was used.

ECG and echocardiography are recorded at every step. The heart rate of an anesthetized rabbit at rest was 203±22 (n=61 tests), 280+31 (n=59) at 40 μg/kg/min, and 299±19 (n=29) at 80 μg/kg/min. The decision to go from 40 to 80 was taken only if the heart rate was below 300 bpm at 40 μg/kg/min.

In conclusion, this test was used to enlighten the behavior of the myocardium during a chemical stress, therefore unveiling an ischemic section of the heart.

### Example 19

### Histological Analysis—Detection of the Atherosclerotic Plaques, Tolerance and FGF-1 Expression

The circonflex coronary artery of two rabbits is examined for the presence of atherosclerotic plaques at the end of the experiment. The hearts are removed and a sample of the left ventricle containing the upper part of the circumflex coronary artery dissected and dipped in PBS buffered 3.7% formalin for further analysis. Each sample is embedded in paraffin. 5 μm sections are performed every 300 μm and stained with Hematoxilin-Eosin-Saffron (HES) for microscopic examination.

Two others rabbits are used to assess the efficiency and safety of a FGF-1 coding plasmid injection in the left ventricular wall. The latest are euthanized at day 3, the heart removed and washed, and the anterior wall observed and kept in formalin for one hour. Then it is divided in five samples, each of them fixed overnight in 3.7% PBS-buffered formalin before being embedded in paraffin.

For each heart, 5 samples presenting macroscopic lesions or supposed to contain injection sites were collected and fixed overnight in 3.7% PBS-buffered formalin before being embedded in paraffin.

A standardized procedure is used for slide preparation. Two 5-μm serial sections are performed from each block. One section is stained with Hematoxylin-Eosin-Saffron (HES) for histopathological examination; the other section is processed for FGF-1 immunohistochemistry (IHC). Injection sites are identified on HES stained sections by the presence of histological changes related to needle and vector injection. The IHC procedure is done using a classical streptavidin-biotin assay. The incubation with a primary polyclonal anti-FGF-1 rabbit antibody (R&D Systems; # AB-32-NA, 1:30 dilution) is followed by incubation with a biotinilated donkey anti-rabbit immunoglobulin (Amersham, 1:200 dilution). The immune complexes are localized using a chromogenic diaminobenzidine substrate, after adding peroxydase coupled to streptavidine. The sections are counterstained with hematoxylin, dehydrated and mounted with permanent mounting media. With this method, immunoreactive fibers appeared brown and the nuclei blue. Previous validation of FGF-1 IHC assay in rabbit muscle demonstrated the ability to detect FGF-1 transgene in myofibers, even when using anti-rabbit secondary antibodies on rabbit tissue samples. Negative control (omitting the primary antibody, but using the secondary antibody) is used to discriminate non-specific staining (mainly extracellular) to specific immunoreactivity. Moreover, the performances of FGF-1 IHC are controlled throughout the assay by using a positive section from a rat muscle having previously demonstrated a high level of FGF-1 expression (slide P1056GNR2, study PAD31.2001). The immunoreactive fibers are identified and counted under a microscope (Zeiss, Axioplan 2). The number of immunoreactive cells given is the number of immunoreactive cardiomyofibers observed around each injection site.

### Example 20

### Demonstration of Therapeutic Angiogenesis in Hypercholesterolemic Settings

A total of 16 Watanabe one-year-old male rabbits were used to assess the efficacy of NV1FGF on reversing myocardial ischemia associated with hypercholesterolemia. In addition, two New Zealand rabbits underwent the same surgical procedure and they were sacrificed at day 3 for the expression analysis.

20.1/ FGF-1 Expression Assessment and Histopathological Pattern

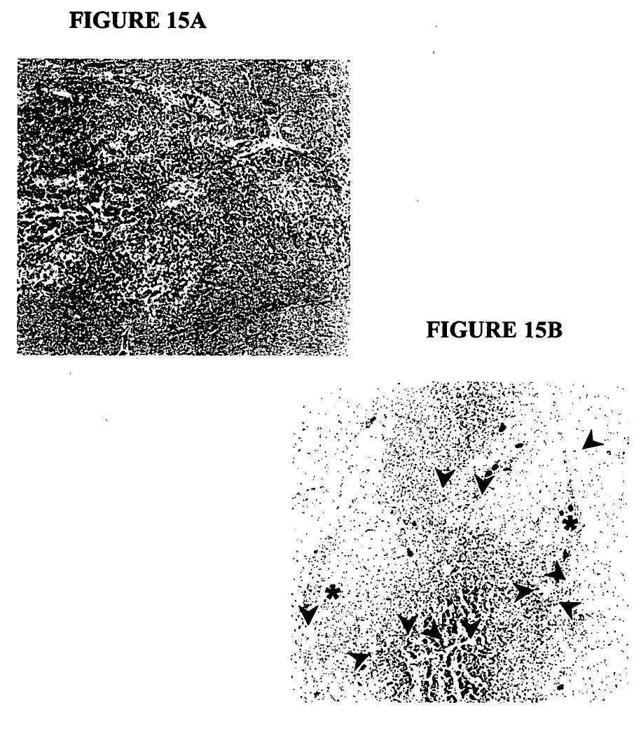

Two healthy New Zealand rabbits were sacrificed three days after the surgical procedure and the injection of NV1FGF. The evaluation of histopathological changes relative to the vector injection was successfully achieved, as 5 and 7 injection sites were localized within the 5 samples analyzed from each heart. Some samples evidenced up to 3 distinct injection sites, separated by 6 to 10 mm, corresponding to the distance between 2 injections. The myocardial lesions were more or less linear and consisted of degeneration and necrosis with active chronic inflammatory response (see FIG. 15A). Some samples displayed diffuse infiltration of lymphocytes below the pericardium, suggesting a limited pericarditis. Individual results are shown in table 3.

Three days after an intramyocardial injection of NV1FGF, FGF-1 immunoreactive myofibers were detected in all the samples displaying injection sites. The expression, restricted to the periphery of the histological lesions, varied from 3 to 36 positive cardiomyofibers per injection site (see FIG. 15B). Individual results were indicated in the table 3.

TABLE 3
Histological observations and FGF-1 expression in healthy rabbit myocardium,
3 days after intramyocardial injections of NV1FGF

| | | | | Number of FGF-1 |
|---|---|---|---|---|
| | | | | immunoreactive |
| | Heart | Sample | | myofibers per |
| Dosing | number | number | Histological observations | injection site |
| NV1FGF | 009R1 | 1 | Sub-epicardial infiltration of inflammatory cells | — |
| 25 μg/injection | | 2 | Sub-epicardial infiltration of inflammatory cells | 16 |
| site | | | Myocardial degeneration and necrosis with | |
| 13 sites/heart | | | inflammatory response - 1 injection site | |
| | | 3 | Myocardial degeneration and necrosis with | 10 |
| | | | inflammatory response - 1 linear injection site | |
| | | 4 | Myocardial degeneration and necrosis with | 6; 17 |
| | | | inflammatory response - 2 distinct injection sites | |
| | | 5 | Myocardial degeneration and necrosis with | 11 |
| | | | inflammatory response - 1 linear injection site | |
| | 009R2 | 1 | None | — |
| | | 2 | Sub-epicardial infiltration of inflammatory cells | — |
| | | 3 | Myocardial degeneration and necrosis with | 4; 23; 8 |
| | | | inflammatory response - 3 distinct injection sites | |
| | | 4 | Myocardial degeneration and necrosis with | 36 |
| | | | inflammatory response - 1 injection site | |
| | | 5 | Myocardial degeneration and necrosis with | 13; 3; 12 |
| | | | inflammatory response - 3 distinct injection sites | |

In conclusion, three days after direct injections of NV1FGF within healthy rabbit myocardium, myofibers degeneration and subsequent inflammatory reaction were observed. At such early time points, histological damages were considered as non specific of NV1FGF, as the severity and the type of the lesions were similar to the one observed in pig myocardium following injection of naked DNA plasmid not encoding for any transgene.

The efficiency of transgene expression in rabbit heart was established for intramyocardial injections of NV1FGF as FGF-1 expression was found in all the samples displaying an injection site. Taking into account the number of immunoreactive myofibers per injection site, the level of expression was similar to the one observed after intramyocardial injection of a same amount of NV1FGF in rat heart, assuming that one injection site in rabbit equals a single injection in rat heart.

20.2/ Electrocardiographic Pattern of the Injected Watanabe Rabbits

16 rabbits were included in this study. Every animal underwent the stress test before the surgery and then every week for four weeks. The highest score observed was plotted for both groups (blue for empty plasmid injected animals, pink for NV1FGF injected animals). As soon as day seven, most of the animals of the NV1FGF group got a lower score, indicating that the ischemia tends to disappear under treatment. On the other hand, most of the animals in the empty treated group remained deeply ischemic (maximum ischemic score of 3).

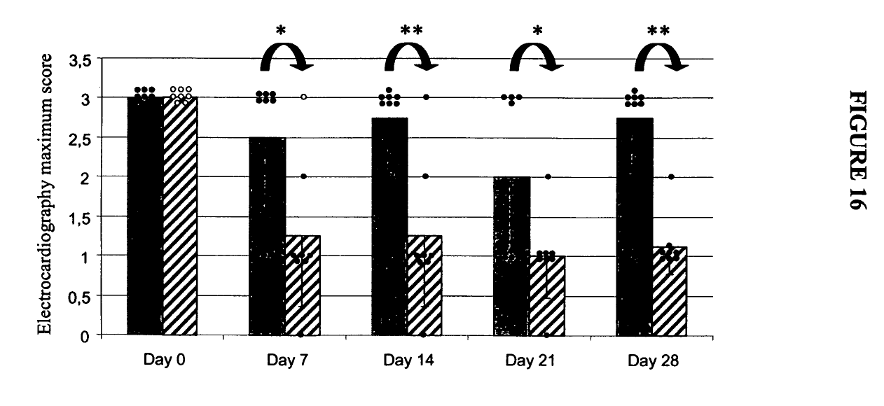

The results as presented in FIG. 16 showed the evolution of the maximum ECG score during the stress test on rabbits treated with empty plasmid (blue dots for individuals, blue column for the mean) or NV1-FGF plasmids (red dots for individuals, pink column for the mean). Treatment of NV1FGF plasmid clearly showed a significant efficacious decrease of the ischemic size.

Starting with day 7, a statistical analysis (unpaired t-test) showed a significant difference between both groups (p<0.05 for *, p<0.01 for **), indicating an effect of the presence of FGF-1 in the regression of the ischemic electrocardiographic pattern at stress.

20.3/ Echocardiographic Pattern of the Injected Watanabe Rabbits

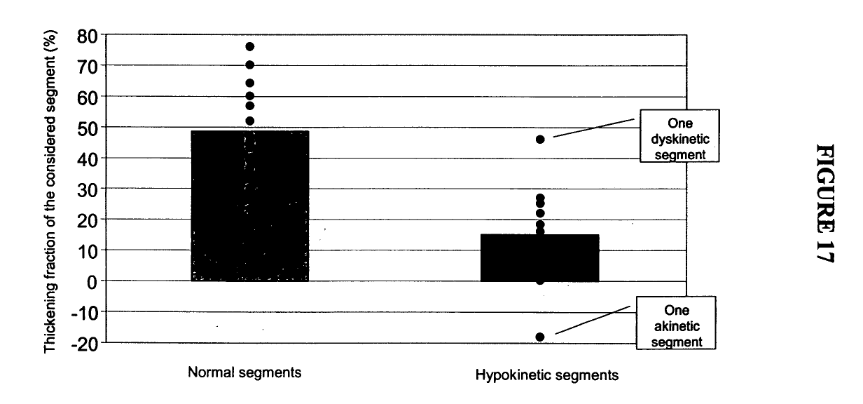

A first step was to validate the accuracy of the correspondence between a qualitative evaluation (classification normal, hypokinesis, akinesis) and the quantitative analysis (fractional wall thickening). 30 segments were analysed, 16 as seen as normal and 14 as abnormal. Their fractional wall thickening was thereafter calculated, and the correspondence was shown in FIG. 17.

As can be seen, the overlap between the two series is very narrow, indicating that the visual evaluation was quite adequate. The abnormal segments included one dyskinetic segment (good thickening fraction but abnormal move) and three akinetic segments, of which one even got thinner during the systole. Subsequently our qualification were considered valid as normal and hypokinetic/akinetic, thus enabling a second and wider evaluation of the myocardial kinetic.

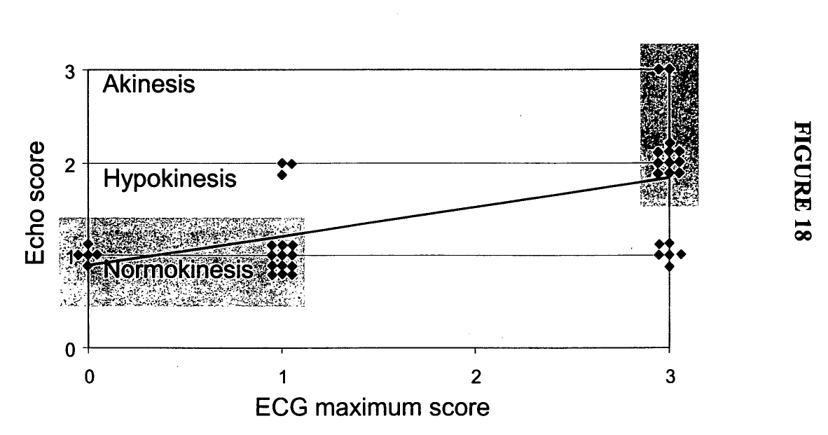

A second step was to correlate the ECG result with the echocardiographic evaluation in each stress test. The result was plotted on FIG. 18. The regression curve was shown in red, indicating that an abnormal ECG was roughly correlated with an abnormal echo. The two shaded areas represented the two main sets of data: the first is the normokinesis echo and normal/slightly ischemic ECG, and the second was the abnormal echo (hypokinesis and akinesis) and the very ischemic ECG (score 3).

The six points where an ischemic ECG corresponded to a normal echo were due to the technical difficulty of getting a good clip for every test, indicating probably that defects on echo were missed. On the other hand, only three tests showed a slightly ischemic ECG with an echo defect. No normal ECG was correlated with an abnormal echo, indicating a good sensitivity for the ECG.

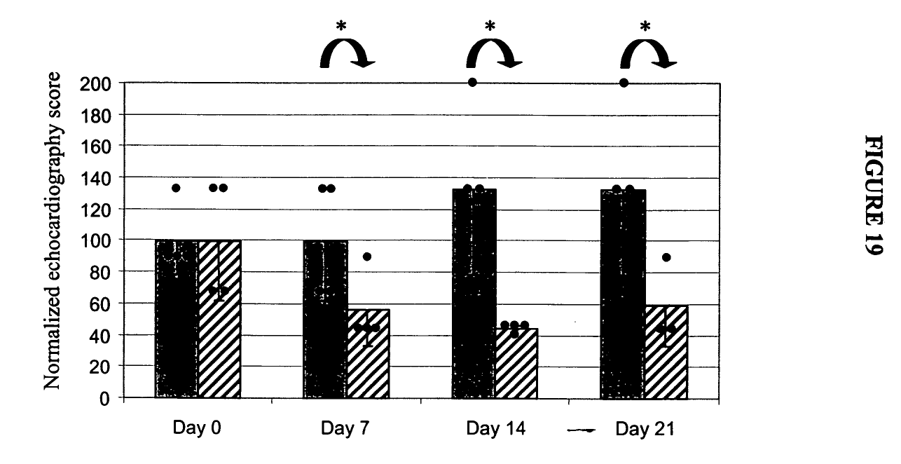

In a third step, evolution of echocardiography in two groups of four treated animals was followed. The score was rated as 1 (normal), 2 (hypokinetic) or 3 (akinetic). No dyskinetic segment was observed in this set of animals. The raw data were plotted in Table 4. As can be seen in this annexe, the baseline values were different (mean of 2.25 versus 1.5). Therefore, the whole set was normalized by using each baseline value as 100%. Then, the following points for each animal were expressed as a percentage of this baseline. The final figure was displayed in FIG. 19.

TABLE 4
raw data of the echocardiography scores

| NV1FGF treated | Day 0 | Day 7 | Day 14 | Day 21 |
|---|---|---|---|---|
| 5063 FGF | 2 | 1 | 1 | 1 |
| 5056 FGF | 3 | 1 | 1 | 2 |
| 5172 FGF | 2 | 2 | 1 | |
| 5171 FGF | 2 | 1 | 1 | 1 |
| Mean | 2.25 | 1.25 | 1.00 | 1.33 |
| SD | 0.50 | 0.50 | 0.00 | 0.58 |
| Empty treated | Day 0 | Day 7 | Day 14 | Day 21 |
| 5061 empty | 1 | 2 | 2 | 1 |
| 5173 empty | 2 | 1 | 1 | 2 |
| 5162 empty | 1 | 2 | 2 | 2 |
| 5302 empty | 2 | 1 | 3 | 3 |
| Mean | 1.50 | 1.50 | 2.00 | 2.00 |
| SD | 0.58 | 0.58 | 0.82 | 0.82 |

The NV1FGF treated animals score was significantly lower than the empty plasmid treated animals score (analysis performed by unpaired t-test), indicating an effect of the presence of FGF-1 on the myocardium contractility, while the empty plasmid treated animals score increases.

Of note, as the final sacrifice step included other technical analysis was not detailed here, no echo recording was available for these animals at day 28.

The results as presented in FIG. 19 clearly showed that treated animals with multiple NV1FGF injections in the myocardium lead to a regression of the ischemia pattern at stress both in ECG and echocardiography, while the empty plasmid injection did not. These results also clearly showed that NV1FGF effects helped the heart to adapt to stress conditions in hypercholesteromic settings.

### Example 21

### Demonstration of the Induction of Arterioles in NV1FGF Treated Cardiac Muscle

21.1/ Animals

Adult male mini-swine (30 kg) were used for the study. A total of 34 animals (n=8 final expected in each treated and control groups, 4 group total) were used. Animals were housed under standard conditions and fed a regular diet. The Animal Care and Use Committee of Duke University approved all procedures and protocols. Animals received humane care in compliance with the “Principles of Laboratory Animal Care” formulated by the National Society for Medical Research and the “Guide for the Care and Use of Laboratory Animals” prepared by the Institute of Laboratory Animal Resources and published by the National Institutes of Health (NIH publication 85-23, revised 1985).

Animals underwent anesthesia and orotracheal intubation. Continuous electrocardiographic and pulse-oximetric monitoring were used throughout the procedure to ensure a stable cardiac rhythm and oxygenation. Under sterile conditions, a left anterolateral thoracotomy was performed through the fourth intercostal space. The pericardium was isolated longitudinally, and the left atrial appendage retracted to allow exposure of the left circumflex (LCx) artery. The proximal LCx was dissected free to allow placement of a hydraulic occluder and 2 mm ultrasonic flow probe (Transonic Systems, Inc., Ithaca, N.Y.) around the vessel. The flow probe was placed distal to the occluder to record downstream flow through the LCx. The occluder and flow probe was then exteriorized through a separate stab incision. A 20 French chest tube was placed and the wound was closed in layers. The chest tube was removed at the conclusion of the procedure. Three days postoperatively, the occluder was inflated to reduce resting blood flow in the LCx to approximately 10% of baseline as assessed using the implanted flow probe. The animals were kept in this low-flow state for two weeks with blood flow recordings being performed three times per week to assure to same degree of vascular occlusion prior to physiologic assessment.

21.2/ Positron Emission Tomography, Dobutamine Stress Echocardiography and Colored Microspheres

After 32±11 days in the low-flow state, the animals underwent positron emission tomography (PET) and dobutamine stress echocardiography (DSE) to characterize the blood flow, metabolic, and functional status of the heart, and document the presence of ischemic, viable myocardium in the LCx distribution. PET scans were interpreted as showing hibernating myocardium if a flow deficit is noted in the lateral and posteroinferior walls of the left ventricle supplied by the LCx accompanied by normal or increased glucose utilization in these same regions (both as compared to the non- ischemic septum). Using DSE, viability in the lateral and posteroinferior walls of the left ventricle was defined as an improvement in systolic wall thickening with low dose dobutamine in myocardial regions with severe hypocontractility at rest. Viable segments were considered ischemic if systolic wall motion was deteriorated with stress (biphasic response).

Dosing was performed after PET and DSE confirm the presence of ischemic myocardium, by direct intramyocardial injection of the FGF expression plasmid such as NV1FGF, with an open chest approach (52±16 days post LCx occlusion). The vectors were administered in 10 sites (100 μg/100 l/injection site for plasmidic vectors) distributed into the free left ventricular wall. 10 injections of 100 μl of saline were performed for the control group. The treatments were performed by operators and investigators which were blinded to the treatment. All efficacy parameters were assigned in a blinded manner, and the code was opened at the end of the study.

21.3/Experimental Design

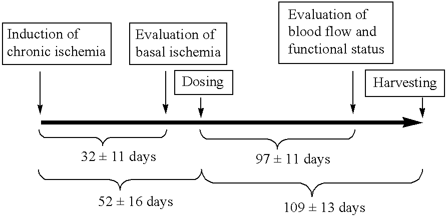

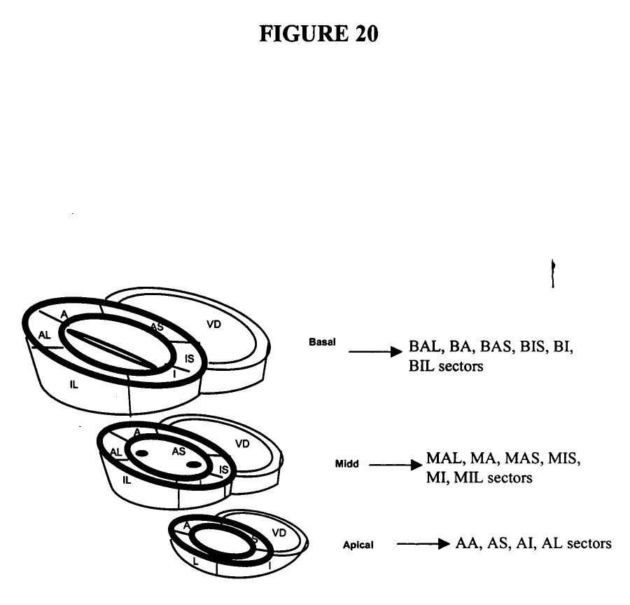

109±13 days after the treatment, the hearts were excised for histologic analysis and perfusion assay. A standardized procedure was used for samples preparation as shown in FIG. 20. More precisely, the hearts were sectioned in 3 short axis slices (apical, mid and basal segments). Each of these segments was subdivided in 6 (mid and basal segments) or 4 orientated sectors (apical segment) giving a total of 16 sectors per heart. Each sector was then divided in 3 transmural samples; one sample was stored for microsphere perfusion analysis, another was snap frozen in liquid nitrogen-cooled isopentane while the latter sample was fixed in 3.7% formalin. A sample from the right ventricle was collected as control.

21.4/ Histological Analysis

A standardized procedure was used for slide preparation. Each formalin-fixed sample was embedded in paraffin wax and three 5-μm serial sections were performed from each block. One section was stained with Hematoxylin-Eosin-Saffron (HES) for evaluation of post-necrotic fibrosis by histopathological examination; the serial section was stained with anti-α Smooth Muscle Actin (SMA) antibody to identify arteries and arterioles. α-SMA is indeed expressed in both pericytes and smooth muscle cells associated with endothelial cells in mature blood vessels (Benjamin et al., Development, 125, 1591-1598. 1998). Of note, some large veins can be stained with this antibody, but are easily identified on morphological criteria.

21.4.1/ α-SMA Staining

All sectors from all the pigs were processed with A-SMA immunohistochemistry. The procedure was done using the Dako EnVision™+/HRP (Horse Radish Peroxidase) detection system (Dako, Sabattini et al., J. Clin Pathol, 51:506-511, 1998). The sections were incubated with an anti α-SMA monoclonal antibody (Dako, clone 1A4, 1:100 dilution). The second step consisted in incubation with goat anti-rabbit antibody conjugated to an HRP labeled polymer. The immune complexes were localized using the chromogenic diaminobenzidine substrate. The sections were counterstained with hematoxylin, dehydrated and mounted with permanent mounting media. With this method immunoreactive cells appeared brown and the nuclei blue.

21.4.2/ Morphometric Analysis

The measurements were performed by a single observer blinded to the treatment regimen. For each sector, the HES-stained section was first analyzed in order to determine the scar area (post necrotic fibrosis). The amount of fibrosis in the sample was scored at low magnification (×25) using the following scale:
+: minimal (less than 5% of the surface of the sample affected by fibrosis)+
++: mild (˜5-15% of the surface of the sample)
+++: marked (˜15-25% of the surface of the sample)
++++: severe (more than 25% of the sample).

Evaluation of vascular density was performed on the serial α-SMA-stained section. The number of α-SMA stained vessels was counted in 9 high-power microscopic fields (0.37 mm² each) located in i) the center of the scar (3 fields), ii) the border of the scar (3 fields) and iii) distant from the scar, i.e. in viable myocardium (3 fields). For each field, 3 categories of vessels were recorded: small unilayered vessels, multilayered vessels with a diameter <100 μm, arterioles and arteries with a diameter >100 μm (see FIG. 2). Large veins with a α-SMA staining were excluded from the analysis, based on their morphological features. Of note, the numerous myofibroblasts containing α-SMA filaments were excluded from the morphometric analysis.

The vascular density (i.e. the number of each category of vessels per mm²) was then calculated for each zone (scar, border zone, viable myocardium) by pooling data from the 5 sectors supposed to be injected (BA, BAL, MA, MAL and AA sectors). To serve as an additional control, vascular density in the non-injected zone (BIL, BI, BIS, BAS, MIL, MI, MIS, MAS, AL, AI, AS sectors) was also calculated.

21.4.3/ Transgene Expression

BA, BAL, MA, MAL and AA sectors from the NV1FGF-treated pigs were processed for FGF-1 immunohistochemistry. FGF-1 expression was assessed using a classical streptavidin-biotin assay. The incubation with the primary polyclonal anti-FGF- 1 rabbit antibody (R&D Systems; # AB-32-NA, 1:30 dilution) was followed by incubation with a biotinilated donkey anti-rabbit immunoglobulin (Amersham, 1:200 dilution). The immune complexes were localized using a chromogenic diaminobenzidine substrate, after adding peroxydase coupled to streptavidine. The sections were counterstained with hematoxylin, dehydrated and mounted with permanent mounting media. With this method, immunoreactive fibers appeared brown and the nuclei blue. The performances of the FGF-1 IHC were controlled throughout the assay by using a positive section from a rat muscle having demonstrated a high level of pCOR- CMV.ratFGF-1 plasmid gene transfer.

21.4.4/ Statistical Analysis

Quantitative variations in vasculature were evidenced in the different zones (scar, border, viable myocardium) between saline and plasmid-treated groups, arithmetical means and standard deviation (Sd) were calculated for each category of vessels. Intergroup data were compared using a one-way analysis of variance (ANOVA, SigmaStat®, Jandel Scientific). When a significant difference was found, multiple comparisons versus control group were performed using the Dunnett's method. All differences were considered to be significant at a level of p<0.05.

21.5/ Evaluation of the Vascular Density

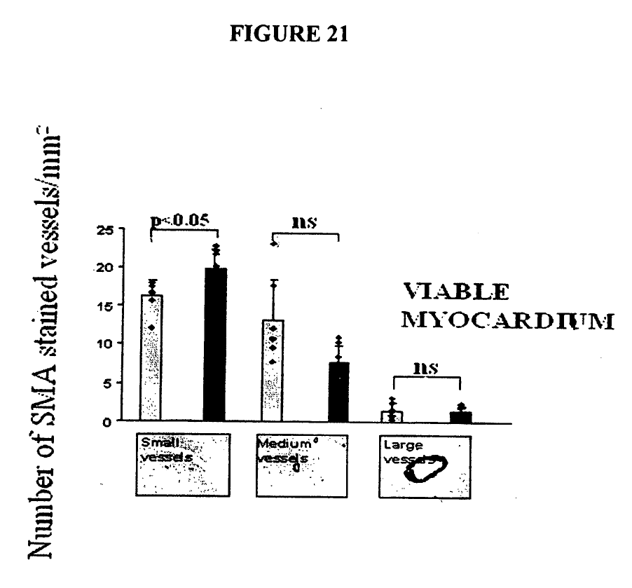

Comparisons of the means were made between the injected zone in the treated animals with NV1FGF and NaCl injected zone in the controls animals (FIG. 21).

Analysis was performed separately for each class of vessels (small, median, large) in the viable myocardium. Whatever the treatment group, numerous arterial structures with a diameter <100 μm were observed in the fibrotic scar. Based on morphological criteria only, these vessels were indistinguishable from native myocardial vessels. Most of them were small, demonstrating a single layer of smooth muscle cells.

When compared to saline injected areas, the administration of NV1FGF to the heart induced, in the viable part of myocardium located within the injected zone, a 22% and 20% increase in the density of small unilayered vessels (p=0.017). This effect was not demonstrated for larger vessels.

These results clearly showed that direct intramyocardial injection of a pCOR plasmid encoding FGF-1 or the spFGF-1 transgene induced, in ischemic pig hearts, a significant increase in the density of unilayered arteries, i.e. arterioles with a single layer of smooth muscle cells. Also, these results demonstrated a long-lasting FGF-1 expression following direct intramyocardial injection a NV1FGF. Indeed, some cardiomyofibers, always located around the injection site, expressed detectable amount of FGF-1 104 to 132 days after the dosing. This long lasting expression is likely in favor of the recruitment and division of precursor cells needed for arteriogenesis and for the maintenance of the induced new vessels. At the time of harvesting, these vessels are believed to be at an early stage of arteriogenesis.

Each of the references that follow are specifically incorporated herein by reference and can be relied on to make and use the invention. In addition, the claims that follow are not intended to limit the scope of the invention and one of skill in the art, understanding this disclosure, can prepare numerous modifications and equivalent aspect of the invention as disclosed or claimed.

### REFERENCES

1. Ouriel K. Peripheral arterial disease. Lancet. 2001;358:1257-1264.
2. Dormandy J A, Rutherford R B. TransAtlantic inter-consensus (TASC): management of peripheral arterial disease (PAD). J Vasc Surg. 2000;31 (suppl. 1, pt 2):S1-S278.
3. Yu H I, Sheu H H, Lai C J, Lee W J, Chen Y T. Endothelial dysfunction in type 2 diabetes mellitus subjects with peripheral artery disease. Int J Cardiol. 2001;78:19-25.
4. Emanueli C., Salis M B, Stacca T, Gaspa L, Chao L, Piana A, Madeddu P. Rescue of impaired angiogenesis in spontaneously hypertensive rats by intramuscular human tissue kallikrein gene transfer. Hypertension. 2001;38:136-141.
5. Duan J, Murohara T, Ikeda H, Katoh A, Shintani S, Sasaki K I, Kawata H., Yamamoto N, Imaizumi T. Hypercholesterolemia inhibits angiogenesis in response to hindlimb ischemia. Nitric oxide-dependent mechanism. Circulation. 2000;102 (suppl III):III-370-III-376.
6. Jang J J, Ho H K V, Kwan H H, Fajardo L F, Cooke J P. Angiogenesis is impaired by hypercholesterolemia. Role of asymmetric dimethylarginine. Circulation. 2000;102:1414-1419.
7. Hirata K, Li T S, Nishida M, Ito H, Matsuzaki M, Kasaoka S, Hamano K. Autologous bone marrow cell implantation as therapeutic angiogenesis for ischemic hindlimb in diabetic rat model. Am. J. Physiol. Heart Circ. Physiol. 2003;284:H66-H70.
8. Ylä-Herttuala S, Kari Alitalo. Nat Med 2003;6:694-701.
9. Ferrara N, Alitalo K. Clinical applications of angiogenic growth factors and their inhibitors. Nat. Med. 1999;5:1359-1364.
10. Isner J M, Asahara T. Angiogenesis and vasculogenesis as therapeutic strategies for postnatal neovascularization. J. Clin. Invest. 1999; 103:1231-1236.
11. Isner J M. Tissue responses to ischemia: local and remote responses for preserving perfusion of ischemic muscle. J. Clin. Invest. 2000;106:615-619.
12. Carmeliet P. Mechanisms of angiogenesis and arteriogenesis. Nat. Med. 2000;6:389-395.
13. Sordello S, Fons P, Malavaud B, Plouet J. Vascular endothelial growth factor family (VEGF). In Comprehensive Vascular Biology and Pathology. Bikfalvi A., editor. 2000. Springer. Paris, France. 322-331.
14. Boilly B, Vercoutter-Edouart A S, Hondermarck H, Nurcombe V, Le Bourhis X. FGF signals for cell proliferation and migration through different pathways. Cytokine Growth Factor Rev. 2000; 11:295-302.
15. Tabata H, Silver M, Isner J M. Arterial gene transfer of acidic fibroblast growth factor for therapeutic angiogenesis in vivo: critical role of secretion signal in use of naked DNA. Cardiovasc. Res. 1997;35:470-479.
16. Nabel E G, Yang Z Y, Plautz G, Forough R, Zhan X, Haudenschild C C, Maciag T, Nabel G J. Recombinant fibroblast growth factor-1 promotes intimal hyperplasia and angiogenesis in arteries in vivo. Nature. 1993;362:844-846.
17. Rosengart T K, Budenbender K T, Duenas M, Mack, C A, Zhang Q X, Isom O W. Therapeutic angiogenesis: a comparative study of the angiogenic potential of acidic fibroblast growth factor and heparin. J. Vasc. Surg. 1997;26:302-312.
18. Comerota A J, Throm R C, Miller K A, Henry T, Chronos N, Laird J, Sequiera R, Kent C K, Bacchetta M, Goldman C, Salenius J-P, Schmieder F A, Pilsudski R. Naked plasmid DNA encoding fibroblast growth factor type 1 for the treatment of end-stage unreconstructible lower extremity ischemia: Preliminary results of a phase I trial. J. Vasc. Surg. 2002;5:930-936.
19. Pu L Q; Jackson S, Lachapelle K J, Arekat Z, Graham A M, Lisbona, R, Brassard R. A persistent hindlimb ischemia model in rabbit. J. Invest. Surg. 1992;7:49-60.
20. Takeshita S, Isshiki T., Sato T. Increased expression of direct gene transfer into skeletal muscles observed after acute ischemic injury in rats. Lab. Invest. 1996;74:1061- 1065.
21. Soubrier F, Cameron B, Manse B, Somarriba S, Dubertret C, Jaslin G, Jung G, Le Caer C, Dang D, Mouvault J M, Scherman D, Mayaux J F, Crouzet J. pCOR: a new design of plasmid vectors for nonviral gene therapy. Gene Ther. 1999;6:1482-1488.
22. Silvestre J S, Mallat Z, Duriez M, Tamarat R, Bureau M F, Scherman D, Duverger N, Branellec D, Tedgui A, Lévy B I. Antiangiogenic effect of interleukin-10 in ischemia- induced angiogenesis in mice hindlimb. Circ. Res. 2000;87:448-452.
23. Lusis A J. Atherosclerosis. Nature. 2000;407:233-241.
24. Dart A M, Chin-Dusting J P F. Lipids and the endothelium. Cardiovasc. Res. 1999;43:308-322.
25. Chen C H, Cartwright J, Jr., Li Z, Lou S, Nguyen H H, Gotto A M, Jr., Henry P D. Inhibitory effects of hypercholesterolemia and ox-LDL on angiogenesis-like endothelial growth in rabbit aortic explants. Essential role of basic fibroblast growth factor. Arterioscler. Thromb. Vasc. Biol. 1997;17:1303-1312.
26. Van Belle E, Rivard A, Chen D, Silver M, Bunting S, Ferrara N, Symes J F, Bauters C, Isner J M. Hypercholesterolemia attenuates angiogenesis but does not preclude augmentation by angiogenic cytokines. Circulation. 1997;96:2667-2674.
27. Morishita R, Sakaki M, Yamamoto K, Iguchi S, Aoki M, Yamasaki K, Matsumoto K, Nakamura T, Lawn R, Ogihara T, Kaneda Y. Impairment of collateral formation in lipoprotein(a) transgenic mice. Therapeutic angiogenesis induced by human hepatocyte growth factor gene. Circulation. 2002;105:1491-1496.
28. Couffinhal T, Silver M, Kearney M, Sullivan A, Witzenbichler B, Magner M, Annex B, Peters K, Isner J M. Impaired collateral vessel development associated with reduced expression of vascular endothelial growth factor in ApoE^{-/-} mice. Circulation. 1999;99:3188-3198.
29. van Royen N, Hoefer I, Bottingen M, Hua J, Grundmann S, Voskuil M, Bode C, Schaper W, Buschmann I, Piek J J. Local monocyte chemoattractant protein-1 therapy increases collateral artery formation in apolipoprotein E-deficient mice but induces systemic monocytic CD11b expression, neointimal formation, and plaque progression. Circ Res 2003;92:218-225.
30. Yamanouchi J, Nishida E, Itagaki S I, Kawamura S, Doi K, Yoshikawa Y. Aortic atheromatous lesions developed in APA hamsters with streptozotocin induced diabetes: a new animal model for diabetic atherosclerosis. 1. Histopathological studies. Exp. Anim. 2000;49:259-266.
31. Yamanouchi J, Takatori A, Itagaki S I, Kawamura S, Yoshikawa Y. APA hamster model for diabetic atherosclerosis. 2. Analysis of lipids and lipoproteins. Exp. Anim. 2000;49:267-274.
32. Simionescu M, Popov D, Sima A, Hasu M, Costache G, Faitar S, Vulpanovici A, Stancu C, Stem D, Simionescu N. Pathobiochemistry of combined diabetes and atherosclerosis studied on a novel animal model. The hyperlipidemic-hyperglycemic hamster. Am. J. Pathol. 1996;148:997-1014.
33. Dimmeler S, Aicher A, Vasa M, Mildner-Rihm C, Adler K, Tiemann M, Rütten H, Fichtlscherer S, Martin H, Zeiher A M. HMG-CoA reductase inhibitors (statins) increase endothelial progenitor cells via the PI3-kinase/Akt pathway. J. Clin. Invest. 2001;108:391-397.
34. Kureishi Y, Luo Z, Shiojima I, Bialik A, Fulton D, Lefer D J, Sessa W C, Walsh K. The HMG-CoA reductase inhibitor simvastatin activates the protein kinase Akt and promotes angiogenesis in normocholesterolemic animals. Nat. Med. 2000;6:1004-1009.
35. Wecksell M B, Winchester P A, Bush H L, Jr., Kent K C, Prince M R, Wang Y I. Cross-sectional pattern of collateral vessels in patients with superficial femoral artery occlusion. Invest. Radiol. 2001;36:422-429.

1. A method of treating myocardial or skeletal angiogenic disorders or defects associated with hypercholesterolemia or diabetes, in a patient suffering therefrom comprising administering thereto an effective amount of a plasmid encoding a fibroblast growth factor, wherein VEGF-A factor expression is not induced in the myocardial or skeletal muscle.

2. A method of treating vascular endothelium dysfunction associated with hypercholesterolemia or diabetes in a patient suffering therefrom comprising administering in skeletal or myocardial muscles of said patient an amount of a plasmid encoding a fibroblast growth factor sufficient to reverse myocardial or skeletal angiogenic defects, wherein VEGF-A factor expression is not induced in said muscle.

3. A method of treating myocardial or skeletal angiogenic disorders associated with hypercholesterolemia or diabetes in a patient suffering therefrom comprising administering thereto an amount of a plasmid encoding a fibroblast growth factor sufficient to promote blood vessels formation in myocardium or skeletal muscle of said patient, wherein VEGF-A factor expression is not induced in said muscle.

4. A method of treating myocardial or skeletal angiogenic disorders associated with hypercholesterolemia or diabetes in a patient suffering therefrom comprising administering thereto an amount of a plasmid encoding a fibroblast growth factor sufficient to promote mature collateral blood vessels and arterioles formation in myocardium or skeletal muscle of said patient.

5. A method of promoting the formation of mature collateral vessels in ischemic cardiac or skeletal muscle tissues in a mammalian subject in need of such treatment comprising injecting said tissues of said subject with an effective amount of a plasmid encoding a fibroblast growth factor, wherein VEGF-A factor expression is not induced in said subject.

6. A method of promoting the formation of both collateral blood vessels and arterioles in ischemic myocardial or skeletal muscle tissues in a mammalian subject in need of such treatment comprising injecting said tissues of said subject with an effective amount of a plasmid encoding a fibroblast growth factor.

7. The method of claim 6, wherein the VEGF-A factor expression is not induced in the myocardial or skeletal muscle of said subject.

8. A method of reversing defects in angiogenesis elicited by hypercholesterolemia or diabetes in a patient suffering therefrom without inducing VEGF-A factor expression, comprising injecting myocardial or skeletal tissues of said patient with an effective amount of a plasmid expressing a fibroblast growth factor to promote the formation of both collateral blood vessels and arterioles.

9. A method of promoting formation of mature large conductance vessels (>150 μm collateral vessels) and small resistance arteries (<50 μm arterioles) in myocardial or skeletal muscles of hypercholesterolemic or diabetic patients comprising injecting said muscles with an effective amount of a plasmid expressing a fibroblast growth factor.

10. The method of claim 9, wherein the VEGF-A factor induction is not induced.

11. The method of any one of claims 1 to 10, wherein the injection of plasmid is performed circularly in skeletal muscles located in the posterior and/or front parts of the thigh and the calf.

12. The method of claim 11, wherein the plasmid is administered by multiple injections around the ischemic site of said muscle.

13. The method of any one of claims 1 to 10, wherein the plasmid is administered to the myocardium of said patient by intracoronary, intramyocardial, transthoracic, pericardial, or epicardial injections.

14. The method of claim 13, wherein the administration to the myocardium is performed by multiple injections around the ischemic site of said cardiac muscle or one single injection.

15. The method of claim 13 or 14, wherein the injection is performed with a catheter.

16. The method of claim 15, wherein the catheter is a needle catheter.

17. The method of any one of claims 1 to 16, wherein the fibroblast growth factor is FGF-1 or acidic fibroblast growth factor.

18. The method of any one of claims 1 to 18, wherein the plasmid further comprises a regulatory sequence, a sequence coding a signal peptide upstream to the FGF-1 gene, a termination signal and a polyadenylation signal downstream to the FGF-1 gene.

19. The method of any one of claims 1 to 19, wherein the regulatory sequence is the CMV promoter, the signal peptide sequence is derived from the peptide sequence of interferon, the polyadenylation signal is derived from that of SV40, and the plasmid encoding FGF-1 is designated NV1FGF.

20. The method of any one of claims 1 to 17, wherein the plasmid is administered in combination with a low molecular weight heparin.

21. A method for improving the intramuscular collateral vessels or arterioles in a subject in need of a treatment for myocardial or skeletal angiogenic disorders or defects associated with hypercholesterolemia or diabetes, comprising administering to the subject an effective amount of a plasmid capable of causing the expression of an FGF-1 growth factor, and measuring the change in myocardial or skeletal muscle, whereby one of the following is observed compared to control: the echocardiographic score is reduced; the echocardiographic pattern is returned to normal kinesis; an increase in the density of unilayered arteries; or the angiographic score is increased.

22. The method of claim 21, wherein the administration is to heart muscle tissue.

23. The method of claim 22, where multiple injections to ischemic sites of the heart are employed to administer the plasmid.

24. The method of one of claims 21, wherein the subject has measurable coronary atherosclerotic plaque covering about 15% of the lumen in at least one of the coronary vessels.

25. The method of claim 21, wherein the observed changed compared to control is taken from the scar border of one of more ischemic sites.

26. The method of claim 21, wherein the plasmid is NV1FGF.

27. The method of claim 21, wherein the plasmid is present in an aqueous solution.

28. The method of one of claims 21 to 27, further comprising administering low molecular weight heparin to the subject.
